(19) United States
(12) Patent Application Publication
(10) Pub. No.: US 2007/0299062 A1
(43) Pub. Date: Dec. 27, 2007

(54) Melanin concentrating hormone antagonists

(75) Inventors: Xiufeng Eric Hu, Cincinnati, OH (US); Namal Chithranga Warshakoon, Mason, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company, Cincinnati, OH (US)

(21) Appl. No.: 11/821,960

(22) Filed: Jun. 26, 2007

Related U.S. Application Data
(60) Provisional application No. 60/816,467, filed on Jun. 26, 2006.

(51) Int. Cl. A61K 31/541; A61K 31/5377; A61K 31/519; C07D 498/02

(52) U.S. Cl. 514/227.8; 514/260.1; 514/234.5; 514/252.16; 544/60; 544/114; 544/278

(57) ABSTRACT

The present invention relates to compounds capable of serving as moderators of human and mammalian appetite and as such provide a means for reducing body mass and controlling obesity.

### CROSS REFERENCE TO RELATED APPLICATIONS

This application claims the benefit of U.S. Provisional Application No. 60/816,467, filed on 26 Jun. 2006.

### FIELD OF THE INVENTION

The present invention relates to compounds capable of serving as moderators of human and mammalian appetite and as such provides a means for reducing body mass. The compounds of the present invention are selective against melanin concentrating hormone and exhibit reduced, if any, side effects versus several other compounds which interact with other appetite related brain receptors.

### BACKGROUND OF THE INVENTION

It has been reported that perhaps 50% of the occidental population and 20% of the oriental population are obese (>20% increase over ideal body mass). In fact, obesity and those having an overweight condition may have reached epidemic proportions in the United States and Western Europe. The Surgeon General of the United States estimated that in 1999, 61% of adults were overweight or obese and this number might be as high as 13% for children and adolescents.

In addition to the aesthetic reasons for maintaining a proper weight, obesity may have a deleterious effect on human health. Excessive body mass has been directly correlated to numerous disease states, inter alia, heart disease, cancer, and type II diabetes.

3-(2-Aminoethyl)-1H-indol-5-ol (serotonin) is a chemical responsible for the regulation of a wide range of CNS brain activity. As a result, extensive research has been conducted in order to understand the role of serotonin and the serotonin (5-HT receptor) in the regulation of a variety of brain-regulated physiological processes from depression to appetite control.

Pharmacologists have long known that direct activation of some 5-HT receptors reduces food consumption (G. Curzon et al., Trends Pharmacol Sci, 13, 21-25 (1998)). For example, mutant mice that lack the 5-HT_{2C} receptor are obese and activating this receptor in normal rats decreases their eating behavior. One means for treating obesity in humans was the use of fenfluramine in combination with phentermine (fen-phen). However, it was discovered in July 1997 that patients reportedly taking fen-phen developed heart valve disease and fenfluramine was subsequently voluntarily withdrawn from the market.

Following the discovery in 1996 that melanin concentrating hormone (MCH) affects rodent feeding, researchers isolated an orphan G-protein coupled receptor that binds MCH with a high affinity. It is now established that body weight is regulated by both the central nervous system and the peripheral nervous system. Appetite and the associated cravings are CNS controlled while metabolism of food and energy expenditure are peripheral endocrine actions. It is now believed that antagonism of one melanin concentrating hormone receptor (MCH-1R) leads directly to reduction in obesity via reduction in both the desire for food (satiety) and changes in the metabolism of caloric intake (i.e. formation of fat tissue, glycogen conversion, and rate of energy expenditure).

There is therefore a long felt need for a chemical composition of matter which provides a means for controlling appetite and therefore is capable of reducing obesity in humans, said compound acting selectively as a MCH antagonist, yet having a low affinity for the 5-HT receptors.

### SUMMARY OF THE INVENTION

Compounds of the present invention are effective in controlling appetite, and therefore, obesity and other appetite related disorders. It is also a surprising discovery that the compounds of the present invention have high affinity for MCH-R1 receptors but display low or marginal affinity for 5-HT_{2c} receptors.

The present invention encompasses three major aspects, each having certain categories, aspects, iterations, and specific iterative examples. The major aspects of the present invention include:
i) novel compositions of matter which are selective antagonists for MCH-R1 receptors over 5-HT_{2c} receptors;
ii) compositions or pharmaceutical compositions (matrices) comprising said compositions of matter, and
iii) methods for controlling, abating, preventing, or alleviating the symptoms of diseases or disease states which are controllable by administration of said compositions of matter to a human or mammal, whether said composition of matter is administered alone or in a composition or within a pharmaceutical composition (matrix).

The first major aspect of the present invention as a whole, relates to compounds, which include all enantiomeric and diastereomeric forms and pharmaceutically acceptable salts thereof, said compounds having the formula; 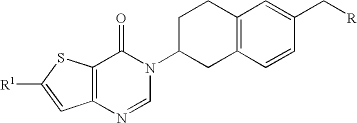
wherein R has the formula: 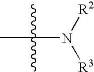
R² and R³ are independently chosen from:
i) hydrogen;
ii) C₁-C₄ substituted or unsubstituted alkyl; or
iii) R² and R³ are taken together to form a substituted or unsubstituted ring containing from 3 to 7 atoms;
   R¹ is a unit chosen from:
i) C₆ or C₁₀ substituted or unsubstituted aryl ring; or
ii) C₃-C₅ substituted or unsubstituted heteroaryl rings.

The second major aspect of the present invention relates to pharmaceutical compositions said compositions comprising:
a) an effective amount of one or more melanin concentrating hormone antagonists according to the present invention; and
b) one or more pharmaceutically acceptable excipients.

The third major aspect of the present invention relates to methods of use. As described herein below, the compounds of the present invention are effective in controlling appetite in humans or higher mammals, and therefore can serve to control, abate, resolve, or otherwise be used to treat one or more diseases or disease states related to food intake, especially obesity and the diseases which are related to or otherwise caused by or induced by obesity, all of which is accomplished without stimulating CNS or peripheral activity caused by activation of one or more 5-HT_{2c} receptors.

The three major aspects of the present invention encompass the discovery that compounds of the present invention, in addition to selectivity as MCH-R1 antagonists, have improved cellular potency and pharmacokinetic properties. This advantage is further exploited in providing a method for controlling obesity and subsequent weight management after weight loss, said method comprising the step of administering to a human or higher mammal an effective amount of a composition comprising one or more of the melanin concentrating hormone antagonists according to the present invention.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to the present invention.

### DETAILED DESCRIPTION OF THE INVENTION

The present invention relates to the surprising discovery that certain compounds (compositions of matter, analogs) bind selectively as antagonists to the MCH-R1 receptor without substantial binding to the 5-HT_{2c} receptor. What is meant herein by “selective binding” is binding to the MCH-R1 receptor at a level at least about 10 fold greater than at the 5-HT_{2c} receptor. For example, a compound with an IC-50 at MCH-R1 of 12 nM and an IC-50 at 5-HT_{2c} of 1125 nM would be a compound which is a selective antagonist at the MCH-R1 receptor over the 5-HT_{2c} receptor.

For the purposes of the present invention the following definitions which are consistent with the usage by the artisan of ordinary skill are used throughout the specification to particularly point out and distinctly claim the subject matter of the present invention.
A. Unsubstituted C₁-C₆ linear, branched, or cyclic alkyl includes but is not limited to the following: methyl (C₁), ethyl (C₂), n-propyl (C₃), iso-propyl (C₃), n-butyl (C₄), sec-butyl (C₄), iso-butyl (C₄), tert-butyl (C₄), and the like.
B. Substituted C₁-C₆ linear, branched, or cyclic alkyl includes but is not limited to the following non-limiting examples: hydroxymethyl (C₁), chloromethyl (C₁), trifluoromethyl (C₁), aminomethyl (C₁), 1-chloroethyl (C₂), 2-hydroxyethyl (C₂), 1,2-difluoroethyl (C₂), 3-carboxypropyl (C₃), and the like.
C. Unsubstituted C₆ or C₁₀ aryl rings are phenyl and naphthyl rings, i.e. phenyl (C₆), naphthylen-1-yl (C₁₀), and naphthylen-2-yl (C₁₀).
D. Substituted C₆ or C₁₀ aryl rings include, but are not limited to 4-fluorophenyl (C₆), 2-hydroxyphenyl (C₆), 3-methylphenyl (C₆), 2-amino-4-fluorophenyl (C₆), 2-(N,N-diethylamino)phenyl (C₆), 2-cyanophenyl (C₆), 2,6-di-tert-butylphenyl (C₆), 3-methoxyphenyl (C₆), 8-hydroxynaphthylen-2-yl (C₁₀), 4,5-dimethoxynaphthylen-1-yl (C₁₀), and 6-cyano-naphthylen-1-yl (C₁₀).
E. Heteroaryl rings which comprise one category of R¹ units as further defined herein include but are not limited to:
   i) 1,2,3,4-tetrazol-1-yl and 1,2,3,4-tetrazol-5-yl having the respective formulae: 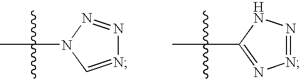
   ii) [1,2,3]triazol-4-yl, [1,2,3]triazol-5-yl, [1,2,4]triazol-4-yl, and [1,2,4]triazol-5-yl having the respective formulae: 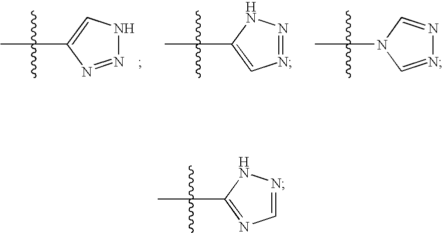
   iii) imidazol-2-yl and imidazol-4-yl having the respective formulae: 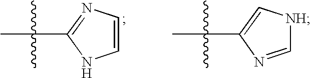
   iv) pyrrol-2-yl and pyrrol-3-yl having the respective formulae: 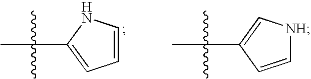
   v) oxazol-2-yl, oxazol-4-yl, and oxazol-5-yl having the respective formulae: 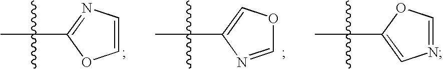
   vi) isoxazol-3-yl, isoxazol-4-yl, and isoxazol-5-yl having the respective formulae: 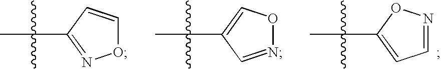
   vii) [1,2,4]oxadiazol-3-yl and [1,2,4]oxadiazol-5-yl having the respective formulae: 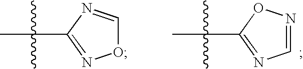
   viii) [1,3,4]oxadiazol-2-yl having the formula: 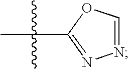
   ix) furan-2-yl and furan-3-yl having the respective formulae: 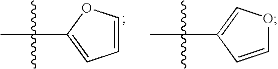
   x) thiophene-2-yl and thiophene-3-yl having the respective formulae: 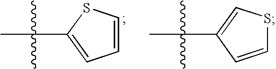
   xi) isothiazol-3-yl, isothiazol-4-yl and isothiazol-5-yl having the respective formulae: 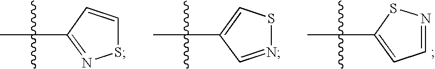
   xii) thiazol-2-yl, thiazol-4-yl and thiazol-5-yl having the respective formulae: 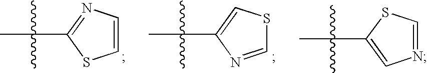
   xiii) [1,2,4]thiadiazol-3-yl and [1,2,4]thiadiazol-5-yl having the respective formulae: 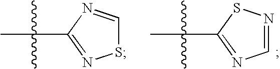
   xiv) [1,3,4]thiadiazol-2-yl having the formula: 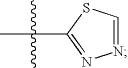
   xv) pyridin-2-yl, pyridin-3-yl, and pyridin-4-yl having the respective formulae: 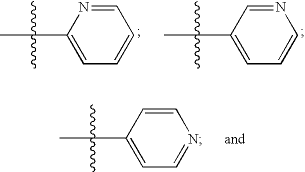
   xvi) pyrimidin-2-yl, pyrimidin-4-yl, and pyrimidin-5-yl having the respective formulae:

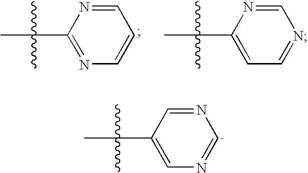

The term “substituted” is used throughout the specification. The term “substituted” is defined herein as “a carbon and hydrogen-containing moiety, which has one or more hydrogen atoms replaced by a substituent or several substituents as defined herein below.” Non-limiting examples of such carbon and hydrogen-containing moiety include hydrocarbyl and heteroaryl moieties, each being acyclic or cyclic, linear or branched. The units, when substituting for hydrogen atoms are capable of replacing one hydrogen atom, two hydrogen atoms, or three hydrogen atoms of a hydrocarbyl moiety at a time. In addition, these substituents can replace two hydrogen atoms on two adjacent carbons to form said substituent, new moiety, or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. Three hydrogen replacement includes cyano, and the like. The term substituted is used throughout the present specification to indicate that a hydrocarbyl moiety, inter alia, aromatic ring, alkyl chain; can have one or more of the hydrogen atoms replaced by a substituent. When a moiety is described as “substituted” any number of the hydrogen atoms may be replaced. For example, 4-hydroxyphenyl is a “substituted aromatic carbocyclic ring”, (N,N-dimethyl-5-amino)octanyl is a “substituted C₈ alkyl unit, 3-guanidinopropyl is a “substituted C₃ alkyl unit,” and 2-carboxypyridinyl is a “substituted heteroaryl unit.”

The following are non-limiting examples of categories and examples herewith of units which can suitably substitute for hydrogen atoms on an alkyl, cycloalkyl, heterocyclic, aryl, or heteroaryl unit described herein below.
i) —NHCOR⁵; for example, —NHCOCH₃, —NHCOCH₂CH₃, —NHCOC₆H₅;
ii) —COR⁵; for example, —COCH₃, —COCH₂CH₃, —COCH₂CH₂CH₃;
iii) —CO₂R⁵; for example, —CO₂CH₃, —CO₂CH₂CH₃, —CO₂CH₂CH₂CH₃;
iv) —OCOR⁵; for example, —OCOCH₃, —OCOCH₂CH₃, —OCOCH₂CH₂CH₃;
v) —C(═NH)NH₂;
vi) —NHC(═NH)NH₂;
vii) —N(R⁵)₂; for example, —NH₂, —NHCH₃, —N(CH₃)₂, —NH(CH₂CH₃);
viii) —NHC₆H₅;
ix) C₁-C₄ linear, branched, or cyclic alkyl; for example, methyl, ethyl;
x) —CON(R⁵)₂; for example, —CONH₂, —CONHCH₃, —CON(CH₃)₂;
xi) —CONHNH₂;
xii) —NHCN;
xiii) —CN;
xiv) halogen: —F, —Cl, —Br, and —I;
xv) —NHN(R⁵)₂; for example, —NHNH₂, —NHNHCH₃, —NHN(CH₃)₂;
xvi) —OR⁵; for example, —OH, —OCH₃, —OCH₂CH₃, —OCH₂CH₂CH₃;
xvii) —NO₂;
xviii) —CHₘXₙ; wherein X is halogen, m is from 0 to 2, m+n=3; for example, —CH₂F, —CHF₂, —CF₃, —CCl₃, or —CBr₃;
xix) —SO₂N(R⁵)₂; for example, —SO₂NH₂; —SO₂NHCH₃; —SO₂NHC₆H₅; and
xx) —SO₂R⁵; for example, —SO₂H; —SO₂CH₃; —SO₂C₆H₅.

For the purposes of the present invention the terms “compound” and “analog” stand equally well for the novel compositions of matter described herein, including all enantiomeric forms, diastereomeric forms, salts, and the like, and the terms “compound” and “analog” are used interchangeably throughout the present specification.

Melanin Concentrating Hormone Antagonists

The compounds of the present invention are melanin concentrating hormone antagonists and comprise all enantiomeric and diastereomeric forms and pharmaceutically acceptable salts thereof, said antagonists having the principle 6-substituted-3-(6-substituted-methyl-1,2,3,4-tetrahydronaphthalen-2-yl)-3H-thieno[3,2-d]pyrimidin-4(3H)-one scaffold with the formula: 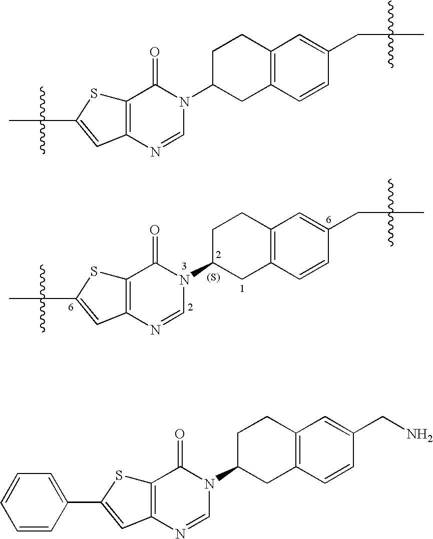
3-(6-aminomethyl-1,2,3,4-tetrahydronaphthalen-2-yl)-6-phenyl-3H-thieno[3,2-d]pyrimidin-4(3H)-one

R is a unit having the formula: 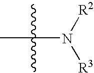
wherein R₂ and R₃ are independently chosen from:
i) hydrogen;
ii) C₁-C₄ substituted or unsubstituted alkyl; or
   R² and R³ are taken together to form a substituted or unsubstituted ring containing from 3 to 7 atoms.

The first category of R units relates to units wherein R² and R³ are each independently hydrogen, methyl, or ethyl, said R units chosen from:
i) —NH₂;
ii) —NHCH₃;
iii) —N(CH₃)₂;
iv) —NHCH₂CH₃;
v) —N(CH₃)(CH₂CH₃); and
vi) —N(CH₂CH₃)₂.

The second category of R units relates to units wherein R² and R³ are each independently hydrogen, n-propyl, iso-propyl, n-butyl, and iso-butyl.

The third category of R units relates to units R² and R³ are taken together to form a ring containing from 3 to 7 atoms.

The first aspect of the third category of R units relates to R² and R³ units taken together to form a ring chosen from aziridin-1-yl, azetidin-1-yl, pyrrolidin-1-yl, piperidin-1-yl, piperazin-1-yl, morpholin-4-yl, 1,1-dioxo-1λ⁶-thiomorpholin-4-yl, and 3,6-diazabicyclo[3.1.1]hept-3-yl.

The second aspect of the third category of R units relates to R² and R³ units taken together to form a substituted ring chosen from aziridin-1-yl, azetidin-1-yl, pyrrolidin-1-yl, piperidin-1-yl, piperazin-1-yl, morpholin-4-yl, and 3,6-diazabicyclo[3.1.1]hept-3-yl, said substitution chosen from:
i) —NHCOR⁴;
ii) —COR⁴;
iii) C₁-C₄ linear, branched, or cyclic alkyl;
iv) —OR⁴;
v) —SO₂R⁴; and
vi) a heterocyclic ring chosen from pyrrolidin-1-yl, piperidin-1-yl, and morpholin-4-yl;
   R⁴ is hydrogen, methyl, ethyl, iso-propyl, and phenyl. Non-limiting examples of the second aspect of the third category of R units include 3-hydroxypyrrolidin-1-yl, 4-methanesulfonylpiperidin-1-yl, 4-acetylpiperidin-1-yl, 4-methylpiperidin-1-yl, 4-(morpholin-4-yl)piperidin-1-yl, 2-oxo-piperidin-1-yl, 4-methanesulfonylpiperazin-1-yl, 4-acetylpiperazin-1-yl, 4-methylpiperazin-1-yl, 4-(morpholin-4-yl)piperazin-1-yl, and 6-methyl-3,6-diazabicyclo[3.1.1]hept-3-yl.

R¹ is a unit chosen from:
i) C₆ or C₁₀ substituted or unsubstituted aryl ring; or
ii) C₂-C₅ substituted or unsubstituted heteroaryl rings.

The first category of R¹ units relates to phenyl and substituted phenyl units (C₆ aryl), the first aspect of which relates to R¹ units which are phenyl or phenyl substituted by halogen which includes units chosen from phenyl, 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2,3,4-trifluorophenyl, 2,3,5-trifluorophenyl, 2,3,6-trifluorophenyl, 2,4,5-trifluorophenyl, 3,4,5-trifluorophenyl, 2,4,6-trifluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, and 2,4,6-trichlorophenyl.

The second aspect of the first category of R¹ units relates to substituted C₆ aryl units which are substituted with halogen substituted alkoxy units, non-limiting examples of which include 2-fluoromethoxyphenyl, 3-fluoromethoxyphenyl, 4-fluoromethoxyphenyl, 2-difluoromethoxyphenyl, 3-difluoromethoxyphenyl, 4-difluoromethoxyphenyl, 2-trifluoromethoxyphenyl, 3-tri-fluoromethoxyphenyl, and 4-trifluoromethoxyphenyl

The third aspect of the first category of R¹ units relates to substituted C₆ aryl units which includes units chosen from 2-methylphenyl, 3-methylphenyl, 4-methylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 3,4,5-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2,4,5-triethylphenyl, 3,4,5-triethylphenyl, and 2,4,6-triethylphenyl.

The fourth aspect of the first category of R¹ units relates to substituted C₆ aryl units, which includes units chosen from 2-methoxyphenyl, 3-methoxyphenyl, 4-methoxyphenyl, 2,3-dimethoxyphenyl, 2,4-dimethoxyphenyl, 2,5-dimethoxyphenyl, 2,6-dimethoxyphenyl, 3,4-dimethoxyphenyl, 3,5-dimethoxyphenyl, 2,3,4-trimethoxyphenyl, 2,3,5-trimethoxyphenyl, 2,3,6-trimethoxyphenyl, 2,4,5-trimethoxyphenyl, 2,4,6-trimethoxyphenyl, 2-hydroxyphenyl, 3-hydroxyphenyl, 4-hydroxyphenyl, 2,3-dihydroxyphenyl, 2,4-dihydroxyphenyl, 2,5-dihydroxy-phenyl, 2,6-dihydroxyphenyl, 3,4-dihydroxyphenyl, 2,3,4-trihydroxyphenyl, 2,3,5-trihydroxyphenyl, 2,3,6-trihydroxyphenyl, 2,4,5-trihydroxyphenyl, 3,4,5-trihydroxyphenyl, and 2,4,6-trihydroxy-phenyl.

The fifth aspect of the first category of R¹ units relates to substituted C₆ aryl units, which includes units chosen from 2-cyanophenyl, 3-cyanophenyl, 4-cyanophenyl, 2,3-dicyanophenyl, 2,4-dicyanophenyl, 2,5-dicyanophenyl, 2,6-dicyanophenyl, 3,4-dicyanophenyl, 3,5-dicyanophenyl, 2,3,4-tricyanophenyl, 2,3,5-tricyanophenyl, 2,3,6-tricyanophenyl, 2,4,5-tricyanophenyl, 3,4,5-tricyanophenyl, and 2,4,6-tricyanophenyl.

The sixth aspect of the first category of R¹ units relates to substituted C₆ aryl units, which includes units chosen from 2-nitrophenyl, 3-nitrophenyl, 4-nitrophenyl, 2,3-dinitrophenyl, 2,4-dinitrophenyl, 2,5-dinitrophenyl, 2,6-dinitrophenyl, 3,4-dinitrophenyl, 3,5-dinitrophenyl, 2,3,4-trinitrophenyl, 2,3,5-trinitrophenyl, 2,3,6-trinitrophenyl, 2,4,5-trinitrophenyl, 3,4,5-trinitrophenyl, and 2,4,6-trinitrophenyl.

The seventh aspect of the first category of R¹ units relates to substituted C₆ aryl units, which includes units chosen from 3-dimethylaminophenyl, 4-dimethylaminophenyl, 3-diethylaminophenyl, 4-diethylaminophenyl, 3-methylsulfanylphenyl, 4-methylsulfanyl-phenyl, 3-ethylsulfanylphenyl, 4-ethylsulfanylphenyl, 3-propylsulfanylphenyl, and 4-propylsulfanylphenyl.

The second category of R¹ units relates to C₂-C₅ substituted or unsubstituted heteroaryl units, the first aspect of which relates to R¹ units which are halogen substituted or unsubstituted C₅ heteroaryl units which include units chosen from pyridin-2-yl, pyridin-3-yl, pyridin-4-yl, 2-fluoropyridin-3-yl, 4-fluoropyridin-3-yl, 5-fluoropyridin-3-yl, 6-fluoropyridin-3-yl, 2-chloropyridin-3-yl, 4-chloropyridin-3-yl, 5-chloropyridin-3-yl, 6-chloropyridin-3-yl, 2-chloropyridin-4-yl, and 3-chloropyridin-4-yl.

The second aspect of the second category of R¹ units relates to C₅ heteroaryl units which are substituted with C₁-C₄ alkyl or C₁-C₄ alkoxy, non-limiting examples of which include 2-methoxypyridin-3-yl, 6-methoxypyridin-3-yl, 2-methoxypyridin-4-yl, 6-methoxypyridin-4-yl, 2-methylpyridin-3-yl, 6-methylpyridin-3-yl, 2-methylpyridin-4-yl, and 6-methylpyridin-4-yl.

The third aspect of the second category of R¹ units relates to C₄ heteroaryl units, said units chosen from furan-2-yl, furan-3-yl, thiophene-2-yl, and thiophene-3-yl.

The analogs (compounds) of the present invention described herein below are arranged in a manner to assist the formulator in applying a rational synthetic strategy for the preparation of analogs which are not expressly exampled herein. This arrangement does not imply increased or decreased efficacy for any of the compositions of matter described herein.

The following illustrates the manner in which R units of the present invention can be varied. The compounds of Table I have the core scaffold with the formula:

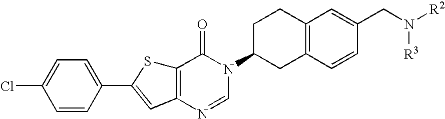

and non-limiting examples of R² and R³ are described therein.

TABLE I

| No. | R² | R³ | No. | R²/R³ rings |
|---|---|---|---|---|
| 1 | —H | —H | 16 | pyrrolidin-1-yl |
| 2 | —CH₃ | —H | 17 | 3-hydroxypyrrolidin-1-yl |
| 3 | —CH₃ | —CH₃ | 18 | piperidin-1-yl |
| 4 | —CH₂CH₃ | —H | 19 | 4-methanesulfonylpiperidin-1-yl |
| 5 | —CH₂CH₃ | —CH₃ | 20 | 4-acetylpiperidin-1-yl |
| 6 | —CH₂CH₃ | —CH₂CH₃ | 21 | 4-methylpiperidin-1-yl |
| 7 | —CH₂CH₂OH | —H | 22 | 4-(morpholin-4-yl)piperidin-1-yl |
| 8 | —CH₂CH₂CH₂OH | —H | 23 | 2-oxo-piperidin-1-yl |
| 9 | —CH₂CH(OH)CH₃ | —H | 24 | piperazin-1-yl |
| 10 | —CH(CH₂OH)₂ | —H | 25 | 4-methanesulfonylpiperazin-1-yl |
| 11 | —CH₂CH₂OH | —CH₃ | 26 | 4-acetylpiperazin-1-yl |
| 12 | —CH₂CH₂CH₂OH | —CH₃ | 27 | 4-methylpiperazin-1-yl |
| 13 | —CH₂CH(OH)CH₃ | —CH₃ | 28 | morpholin-4-yl |
| 14 | —CH(CH₂OH)₂ | —CH₃ | 29 | 3,6-diazabicyclo[3.1.1]hept-3-yl |
| 15 | —CH(CH₃)₂ | —H | 30 | 1,1-dioxo-1λ⁶-thiomorpholin-4-yl |

Scheme I outlines and Example 1 describes the synthesis of a compound according to the present invention and thereby provides a procedure by which the R units of the compounds of the present invention can be varied.

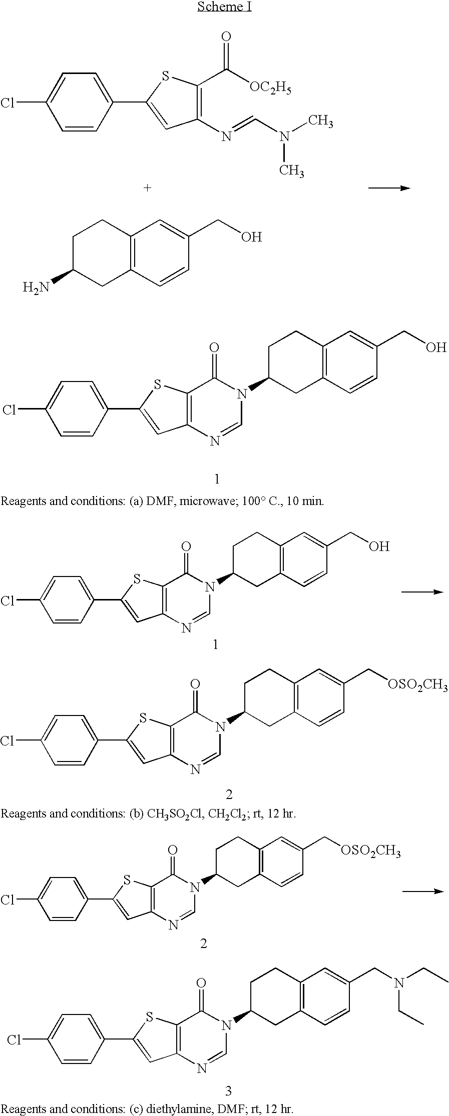

### EXAMPLE 1

### (S)-6-(4-Chlorophenyl)-3-(6-diethylaminomethyl-1,2,3,4-tetrahydronaphthalen-2-yl)-thieno[3,2-d]pyrimidin-4(3H)-one(3)

Preparation of (S)-6-(4-chlorophenyl)-3-(6-(hydroxymethyl)-1,2,3,4-tetrahydronaphthalen-2-yl)thieno[3,2-d]pyrimidin-4(3H)-one (1): (S)-(6-Amino-5,6,7,8-tetrahydronaphthalen-2-yl)methanol (0.50 g, 2.33 mmol), ethyl-5-(4-chlorophenyl)-3-((dimethylamino)-methyleneamino)thiophen-2-carboxylate (0.78 g, 2.33 mmol) and triethylamine (1.63 mL, 11.65 mmol) in DMF (4 mL) are heated in microwave at 100° C. for 10 min. The mixture is diluted with dichloromethane (100 mL), washed with water (5×), brine (1×), dried (Na₂SO₄), and concentrated to afford the desired compound which is taken to the next step without further purification. ¹H NMR (300 MHz, DMSO-d₆): δ 2.00-2.25 (m, 2H), 3.00-3.50 (m, 4H), 4.49 (m, 2H), 5.05 (m, 1H), 5.17 (m, 1H), 7.09-7.15 (m, 3H), 7.63 (m, 2H), 7.93-7.98 (m, 3H), 8.60 (d, J=3.3 Hz, 1H); ¹³C NMR (75 MHz, DMSO-d₆): δ 26.7, 27.1, 29.2, 54.8, 58.1, 124.1, 125.2, 125.8, 126.7, 127.1, 128.5, 129.4, 134.5, 134.8, 135.6, 138.1, 145.1, 148.2, 149.1, 157.1, 159.2; MS (M+1): 423.1.

Preparation of (S)-6-(4-chlorophenyl)-3-(6-methanesulfonylmethyl-1,2,3,4-tetrahydronaphthalen-2-yl)thieno[3,2-d]pyrimidin-4(3H)-one (2): (S)-6-(4-Chlorophenyl)-3-(6-(hydroxymethyl)-1,2,3,4-tetrahydronaphthalen-2-yl)thieno[3,2-d]pyrimidin-4(3H)-one, 1, (0.62 g, 1.46 mmol), methanesulfonyl chloride (0.28 mL, 3.67 mmol) and Et₃N (0.51 mL, 3.67 mmol) are mixed together in dichloromethane (10 mL), stirred for 12 hours and then quenched with water (10 mL). The two layers are allowed to separate, the aqueous layer is extracted with dichloromethane (2×), the organic layers are combined, washed with brine (1×) and evaporated under reduced pressure to afford the desired product as a liquid which solidifies upon standing. This material is taken to the next step without further purification.

Preparation of (S)-6-(4-chlorophenyl)-3-(6-diethylaminomethyl-1,2,3,4-tetrahydronaphthalen-2-yl)thieno[3,2-d]pyrimidin-4(3H)-one (3): (S)-6-(4-chlorophenyl)-3-(6-methanesulfonylmethyl-1,2,3,4-tetrahydronaphthalen-2-yl)-thieno[3,2-d]pyrimidin-4(3H)-one, 2, (0.10 g, 0.22 mmol) and diethylamine (0.50 mL) in DMF (2 mL) are stirred for 12 hours at room temperature. At the completion of the reaction, as indicated by LCMS and HPLC, the reaction is quenched with water (10 mL), extracted with EtOAc (3×20 mL). The combined organic layers are washed with water (5×), brine (1×) and dried (Na₂SO₄). The solvent is removed, and the residue is dissolved in MeOH (2 mL) and purified on HPLC (0.1% TFA/CH₃CN/water). After removal of the solvent, the resultant TFA salts are converted into their HCl salts via stirring them in a methanolic HCl solution. ¹H NMR (300 MHz, DMSO-d₆): δ 1.01 (m, 6H), 2.00-2.21 (m, 2H), 2.45 (m, 4H), 3.00-3.50 (m, 6H), 5.07 (m, 1H), 7.09-7.12 (m, 3H), 7.60 (m, 2H), 7.93-7.95 (m, 3H), 8.61 (d, J=3.3 Hz, 1H); ¹³C NMR (75 MHz, DMSO-d₆): δ 10.9, 14.5, 17.2, 26.7, 27.1, 29.2, 54.8, 60.3, 125.2, 125.8, 126.4, 127.2, 128.2, 129.5, 129.8, 134.2, 134.8, 135.6, 138.2, 145.1, 148.2, 149.1, 157.1; MS (M+1) 478.1.

The following are non-limiting examples of the how the R units of the present invention can be varied.

(S)-6-(4-Chlorophenyl)-3-(6-((4-morpholinopiperidin-1-yl)methyl)-1,2,3,4-tetrhaydronapthalene-2-yl)thieno[3,2,-d]pyrimidin-4-(3H)-one: ¹H NMR (300 MHz, DMSO-d₆): δ 2.16 (s, 2H), 2.34 (d, J=11.7 Hz, 4H), 2.93 (d, J=11.7 Hz, 2H), 3.04 (s, 2H), 3.19-3.27 (m, 5H), 3.41 (d, J=12.0 Hz, 4H), 3.82-3.89 (m, 2H), 3.97-4.01 (m, 2H), 4.25 (s, 2H), 5.02 (m, 1H), 7.09-7.12 (m, 3H), 7.60 (m, 2H), 7.93-7.95 (m, 3H), 8.61 (d, J=3.3 Hz, 1H); ¹³C NMR (75 MHz, DMSO-d₆): δ 23.8, 28.0, 29.4, 35.0, 49.1, 50.0, 51.7, 59.0, 60.3, 63.9, 122.3, 125.2, 125.6, 127.9, 128.6, 129.7, 130.1, 132.0, 132.6, 135.4, 136.8, 145.1, 148.1, 150.4, 156.8, 157.8; MS (M+1) 574.2.

(S)-6-(4-Chlorophenyl)-3-(6-((4-(4-methylpiperazin-1-yl)piperidin-1-yl)methyl)-1,2,3,4-tetrahydronapthalen-2-yl)thieno[3,2-d]pyrimidin-4(3H)-one: ¹H NMR (300 MHz, DMSO-d₆): δ 2.00-2.25 (m, 2H), 2.26-2.54 (m, 8H), 2.64 (s, 3H), 3.00-3.50 (m, 4H), 5.02 (m, 1H), 7.09-7.12 (m, 3H), 7.60 (m, 2H), 7.93-7.95 (m, 3H), 8.61 (d, J=3.3 Hz, 1H); ¹³C NMR (75 MHz, DMSO-d₆): δ 20.4, 24.1, 29.1, 54.1, 54.2, 55.1, 60.3, 122.3, 125.2, 125.6, 127.8, 128.6, 129.7, 130.4, 132.0, 132.6, 135.5, 136.9, 145.1, 148.1, 150.5, 156.9, 157.6; MS (M+1) 505.1.

(S)-6-(4-Chlorophenyl)-3-(6-((4-methylsulfonyl)piperazin-1-yl)methyl)-1,2,3,4-tetrahydronapthalen-2-yl)thieno[3,2,-d]pyrimidin-4(3H)-one: ¹H NMR (300 MHz, DMSO-d₆): δ 2.00-2.25 (m, 2H), 3.02 (s, 3H), 3.20-3.50 (m, 6H), 3.84-3.83 (m, 6H), 4.49 (m, 2H), 5.05 (m, 1H), 7.09-7.12 (m, 3H), 7.60 (m, 2H), 7.93-7.95 (m, 3H), 8.61 (d, J=3.3 Hz, 1H); ¹³C NMR (75 MHz, DMSO-d₆): δ 26.8, 28.8, 33.1, 34.9, 42.8, 51.2, 52.1, 60.1, 122.4, 127.4, 128.6, 129.8, 130.1, 130.3, 132.0, 135.0, 135.1, 148.1, 150.4, 156.8, 157.7; MS (M+1) 569.1.

(S)-6-(Chlorophenyl)-3-(6-(morpholinomethyl)-1,2,3,4-tetrahydronapthalene-2-yl)thieno[3,2-d]pyrimidin-4-(3H)-one: ¹H NMR (300 MHz, DMSO-d₆): δ 1.18-1.23 (m, 4H), 2.30-2.39 (m, 2H), 2.96 (m, 2H), 3.06-3.11 (m, 2H), 3.18-3.26 (m, 2H), 3.57 (m, 4H), 5.02 (m, 1H), 7.09-7.12 (m, 3H), 7.60 (m, 2H), 7.93-7.95 (m, 3H), 8.61 (d, J=3.3 Hz, 1H); ¹³C NMR (75 MHz, DMSO-d₆): δ 8.5, 14.2, 22.1, 29.2, 31.4, 54.7, 54.8, 58.9, 78.1, 122.3, 125.3, 125.4, 126.8, 128.5, 129.8, 130.4, 132.0, 133.6, 135.5, 136.9, 145.1, 148.1, 150.5, 156.9, 157.6; MS (M+1) 492.1.

(S)-3-(6-((4-Acetylpiperazin-1-yl)methyl)-1,2,3,4-tetrahydronapthalen-2-yl)-6-(4-chlorophenyl)thieno[3,2-d]pyrimidine-4(3H)-one: ¹H NMR (300 MHz, DMSO-d₆): δ 2.04 (s, 3H), 2.91-3.37 (m, 12H), 4.04 (m, 1H), 4.31 (s, 2H), 4.46 (m, 1H), 5.02 (m, 1H), 7.09-7.12 (m, 3H), 7.60 (m, 2H), 7.93-7.95 (m, 3H), 8.61 (d, J=3.3 Hz, 1H); ¹³C NMR (75 MHz, DMSO-d₆): δ 20.9, 28.9, 29.9, 35.6, 39.3, 44.0, 52.1, 52.2, 53.2, 61.2, 121.5, 123.5, 127.7, 128.8, 129.9, 130.4, 131.0, 132.7, 136.7, 137.6, 137.9, 147.8, 152.9, 158.1, 158.4, 171.6; MS (M+1) 533.1.

6-(4-Chlorophenyl)-3-((S)-6-(((S)-2-hydroxypropylaminomethyl)-1,2,3,4-tetrahydronapthealen-2-yl)thieno[3,2-d]pyrimidin-4(3H)-one: ¹H NMR (300 MHz, DMSO-d₆): δ 1.10 (d, J=6.34 Hz, 3H), 2.16 (s, 2H), 2.39-2.45 (m, 2H), 2.65-2.68 (m, 2H), 2.86 (s, 1H), 3.00 (s, 1H), 3.11-3.18 (m, 2H), 3.22-3.25 (m, 2H), 5.02 (m, 1H) 7.09-7.12 (m, 3H), 7.60 (m, 2H), 7.93-7.95 (m, 3H), 8.61 (d, J=3.3 Hz, 1H); ¹³C NMR (75 MHz, DMSO-d₆): δ 21.8, 28.1, 29.4, 34.9, 50.4, 51.7, 53.3, 62.9, 122.3, 128.4, 128.6, 129.9, 130.2, 130.3, 132.0, 135.0, 135.1, 148.1, 150.4, 156.8, 157.7; MS (M+1) 480.1.

6-(4-Chlorophenyl)-3-{(S)-6-[((S)-3-hydroxypyrrolidin-1-yl)methyl]-1,2,3,4-tetrahydronaphthalen-2-yl}thieno[3,2-d]pyrimidin-4(3H)-one: ¹H NMR (300 MHz, DMSO): δ 1.28-1.18 (m, 2H), 2.16-1.76 (m, 4H), 3.34-3.01 (m, 7H), 4.29-4.27 (m, 2H), 5.05-5.00 (m, 1H), 7.09-7.12 (m, 3H), 7.60 (m, 2H), 7.93-7.95 (m, 3H), 8.61 (d, J=3.3 Hz, 1H) ; ¹³C NMR (75 MHz, DMSO-d₆): δ 9.1, 28.0, 29.4, 33.1, 33.7, 46.0, 51.7, 60.2, 68.9, 122.3, 128.4, 128.6, 129.9, 130.2, 130.3, 132.0, 135.0, 135.1, 148.1, 150.4, 156.8, 157.7; MS (M+1): 492.10.

(S)-6-(4-Chlorophenyl)-3-(6-(piperidin-1-ylmethyl)-1,2,3,4-tetrahydronaphthalen-2-yl)thieno[3,2-d]pyrimidin-4(3H)-one ¹H NMR (300 MHz, DMSO): δ 8.58 (s, 1H), 7.94 (s, 1H), 7.91 (d, J=8.5 Hz, 2H), 7.58 (d, J=8.5 Hz, 2H), 7.40 (s, 1H), 7.39 (d, J=8.1 Hz, 1H), 7.20 (d, J=8.1 Hz, 1H), 5.05-5.00 (m, 1H), 4.29-4.27 (m, 2H), 3.10-2.55 (series of m, 8H), 1.98-1.90 (m, 2H), 1.18-1.02 (series of m, 6H), LRMS (M+H⁺): 490.51.

(S)-6-(4-Chlorophenyl)-3-(6-(pyrrolidin-1-ylmethyl)-1,2,3,4-tetrahydro-naphthalen-2-yl)thieno[3,2-d]pyrimidin-4(3H)-one: ¹H NMR (300 MHz, DMSO): δ 8.58 (s, 1H), 7.94 (s, 1H), 7.91 (d, J=8.5 Hz, 2H), 7.58 (d, J=8.5 Hz, 2H), 7.40 (s, 1H), 7.39 (d, J=8.1 Hz, 1H), 7.20 (d, J=8.1 Hz, 1H), 5.05-5.00 (m, 1H), 4.29-4.27 (m, 2H), 3.34-3.01 (series of m, 8H), 2.16-1.76 (series of m, 6H); LRMS (M+H⁺): 476.50.

The following illustrate the manner in which the formulator may vary the R¹ units of the present invention. Table II provides non-limiting examples of units which are encompassed within the scope of R¹ units of the present invention.

TABLE II

| No. | R¹ |
|---|---|
| 31 | 2-fluorophenyl |
| 32 | 3-fluorophenyl |
| 33 | 4-fluorophenyl |
| 34 | 2,4-difluorophenyl |
| 35 | 3,4-difluorophenyl |
| 36 | 3,5-difluorophenyl |
| 37 | 2-chlorophenyl |
| 38 | 3-chlorophenyl |
| 39 | 4-chlorophenyl |
| 40 | 2,4-dichlorophenyl |
| 441 | 3,4-dichlorophenyl |
| 42 | 3,5-dichlorophenyl |
| 43 | 2-methylphenyl |
| 44 | 3-methylphenyl |
| 45 | 4-methylphenyl |
| 46 | 2-cyanophenyl |
| 47 | 3-cyanophenyl |
| 48 | 4-cyanophenyl |
| 49 | 2-methoxyphenyl |
| 50 | 3-methoxyphenyl |
| 51 | 4-methoxyphenyl |
| 52 | pyridin-2-yl |
| 53 | pyridin-3-yl |
| 54 | pyridin-4-yl |
| 55 | 2-fluoropyridin-3-yl |
| 56 | 4-fluoropyridin-3-yl |
| 57 | 5-fluoropyridin-3-yl |
| 58 | 6-fluoropyridin-3-yl |
| 59 | 2-chloropyridin-3-yl |
| 60 | 4-chloropyridin-3-yl |
| 61 | 5-chloropyridin-3-yl |
| 62 | 6-chloropyridin-3-yl |
| 63 | 2-methoxypyridin-3-yl |
| 64 | 6-methoxypyridin-3-yl |
| 65 | 2-methoxypyridin-4-yl |
| 66 | 6-methoxypyridin-4-yl |
| 67 | 2-chloropyridin-4-yl |
| 68 | 3-chloropyridin-4-yl |
| 69 | furan-2-yl |
| 70 | furan-3-yl |
| 71 | thiophene-2-yl |
| 72 | thiophene-3-yl |

A convenient starting material useful for variation of R¹ units according to the present invention is 5-bromo-3-(dimethylaminomethyleneamino)thiophene-2-carboxylic acid methyl ester having the formula:

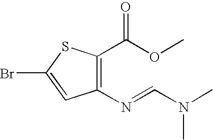

5-Bromo-3-(dimethylaminomethyleneamino)thiophene-2-carboxylic acid methyl ester may be prepared by the following procedure as disclosed in WO 2005/047293.

Preparation of 3-(2,2,2-trifluoracetamido)thiophene-2-carboxylic acid methyl ester: Methyl-3-aminothiophene-2-carboxylate (10.0 g) in acetonitrile (130 mL) is cooled to 0° C. and treated with pyridine (6.2 mL) and trifluoroacetic anhydride (11.7 mL). Stirring is continued for 5 minutes and the reaction mixture is warmed to room temperature and stirred an additional 20 minutes. The reaction is poured into a flask containing ice water (1.5 L) and stirred for 15 minutes. The precipitate which forms is collected by filtration and azeotropically distilled with (3×200 mL) to remove any residual water and affords 15.9 g of the desired compound.

Preparation of 5-bromo-3-(2,2,2-trifluoracetamido)thiophene-2-carboxylic acid methyl ester: To THF (100 mL) at −78° C. is added diisopropylamine (10 mL) and butyllithium (26.4 mL, 2.5 Min hexanes). The reaction mixture is allowed to warm ot 0° C. and stir for 10 minutes. The reaction mixture is re-cooled to −78° C. and 3-(2,2,2-trifluoracetamido)thiophene-2-carboxylic acid methyl ester (5.06 g) dissolved in THF (20 mL) is added via cannula. The reaction is stirred at −78° C. for 1 hour and then treated with 1,2-dibromoethane (10.3) added in one portion. The reaction is stirred an additional 30 minutes at −78° C. then allowed to warm to room temperature for 30 minutes. NaHCO₃ (sat. solution) is added and the aqueous layer extracted with EtOAc (×3). The combined organic layers are washed with water and brine. The organic layer is dried and concentrated under reduced pressure and purified over silica (2.5% EtOAc in hexanes) to afford 2.88 g of the desired product.

Preparation of 3-amino-5-bromothiophene-2-carboxylic acid methyl ester: To a solution of 5-bromo-3-(2,2,2-trifluoracetamido)thiophene-2-carboxylic acid methyl ester in MeOH (45 mL) is added a solution of K₂CO₃ (5.89 g) in water (18 mL). The reaction is stirred for 3 hours after which the solvent is removed under reduced pressure. The resulting crude material is partitioned between EtOAc and water. The organic layer is washed with water, brine, then dried and concentrated under reduced pressure to afford 1.97 g of the desired product.

Preparation of 5-bromo-3-(dimethylaminomethyleneamino)thiophene-2-carboxylic acid methyl ester: To a solution of 3-amino-5-bromothiophene-2-carboxylic acid methyl ester (4.23 g, 17.1 mmol) in EtOH (80 mL) is added dimethylformamide dimethyl acetal (5.9 mL, 44.2 mmol). The reaction is stirred in a pressure vessel at 90° C. for 3 hours. The solvent is removed the excess dimethylformamide dimethyl acetal is removed by co-evaporation with toluene to afford 5.1 g of the desired compound.

Scheme II outlines and Example 2 describes the synthesis of a compound according to the present invention and thereby provides a procedure by which the R¹ units of the compounds of the present invention can be varied.

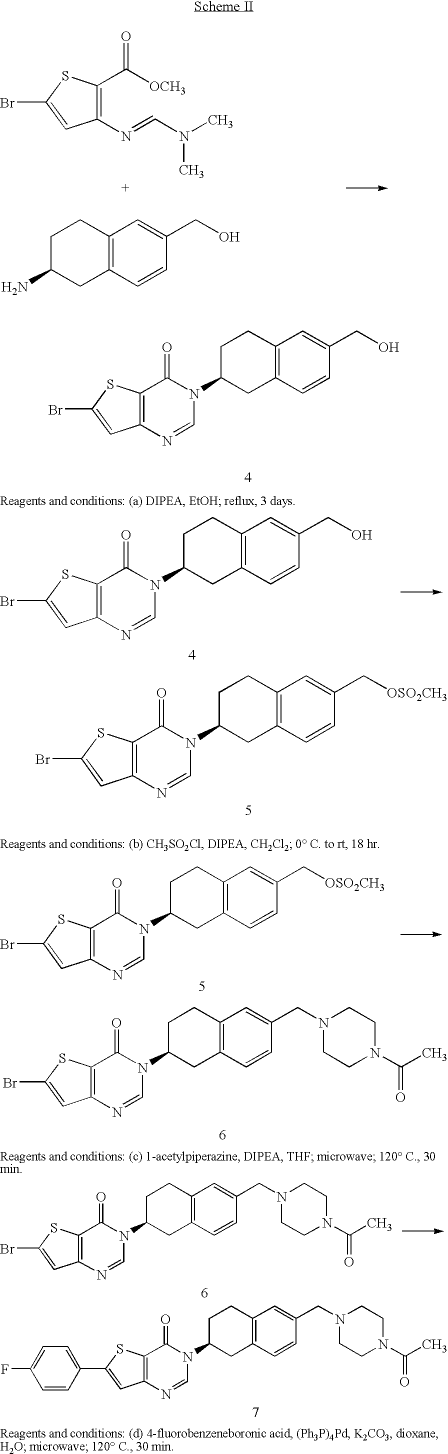

### EXAMPLE 2

### (S)-3-{6-[(4-Acetylpiperazin-1-yl)methyl]-1,2,3,4-tetrahydro-naphthalen-2-yl}-6-(4-fluorophenyl)thieno[3,2-d]pyrimidin-4(3H)-one (7)

Preparation of (S)-6-bromo-3-(6-(hydroxymethyl)-1,2,3,4-tetrahydronaphthalen-2-yl)thieno[3,2-d]pyrimidin-4(3H)-one (4). A suspension of (S)-(6-amino-5,6,7,8-tetrahydronaphthalen-2-yl)methanol (4.35 g, 1.0 eq), 5-bromo-3-(dimethylamino-methyleneamino)thiophene-2-carboxylic acid methyl ester (1.0 eq), and N,N-diisopropylethylamine (2.0 eq) in ethanol (100 mL) is refluxed for 3 days. The reaction is concentrated to dryness. The residue is partitioned between dichloromethane and water. The layers are separated and the aqueous layer extracted twice with dichloromethane. The organic layers are combined, washed with brine, dried over MgSO₄, filtered, and the solvent removed under reduced pressure to afford the desired compound ¹H NMR (300 MHz, CDCl₃): δ 7.84 (s, 1H), 7.34 (s, 1H), 7.21 (s, 1H), 7.19 (d, J=7.7 Hz, 1H), 7.11 (d, J=7.7 Hz, 1H), 5.28-5.18 (m, 1H), 4.68 (s, 2H), 3.28 (dd, J=16.1, 5.5 Hz, 1H), 3.09-2.90 (m, 3H), 2.24-2.16 (m, 2H); ¹³C NMR (75 MHz, CDCl₃): δ 156.4, 156.2, 145.9, 140.1, 135.4, 133.3, 129.7, 128.2, 127.9, 125.6, 125.4, 124.9, 65.3, 51.1, 35.6, 29.1, 29.0

Preparation of 6-bromo-3-(6-methansulfonylmethyl-1,2,3,4-tetrahydronaphthale-2-yl)thieno[3,2-d]pyrimidin-4(3H)-one (5). To a 0° C. solution of (S)-6-bromo-3-(6-(hydroxymethyl)-1,2,3,4-tetrahydronaphthalen-2-yl)thieno[3,2-d]pyrimidin-4(3H)-one, 5, (2.78 g, 1.0 eq.) and N,N-diisopropylethylamine (1.5 eq) in dichloromethane (50 mL) is added a solution of methanesulfonyl chloride (1.2 eq) in dichloromethane (5 mL) drop-wise. After complete addition, the ice bath is removed and the reaction is stirred at room temperature overnight. The reaction mixture is washed with water, and then brine. The aqueous layers are combined and extracted with dichloromethane. The organic layers are combined, dried over MgSO₄, filtered, and the filtrate concentrated to dryness to afford the desired product which is used without further purification.

Preparation of 3-[6-(4-acetylpiperazin-1-ylmethyl)-1,2,3,4-tetrahydronaphthalen-2-yl]-6-bromothieno[3,2-d]pyrimidin-4(3H)-one (6): A suspension of 6-bromo-3-(6-methansulfonylmethyl-1,2,3,4-tetrahydronaphthale-2-yl)thieno[3,2-d]pyrimidin-4(3H)-one, 5, (7.1 mmol), 1-acetylpiperazine (1.2 eq), and N,N-diisopropylethylamine (1.5 eq) in THF (10 mL) is heated in the CEM microwave at 120° C. for 30 min. The solvent is removed under reduced pressure and the residue is dissolved in dichloromethane and washed with water. The organic layers are combined, dried over magnesium sulfate, filtered, and the filtrate concentrated under reduced pressure and the crude material is purified over silica (5% MeOH/CH₂Cl₂) to afford the desired product. ¹H NMR (300 MHz, CDCl₃): δ 8.07 (s, 1H), 7.32 (s, 1H), 7.13-7.01 (m, 3H), 5.29-5.19 (m, 1H), 3.76-3.56 (m, 2H), 3.48 (s, 2H), 3.48-3.45 (m, 2H), 3.30 (dd, J=16.2, 5.5 Hz, 1H), 3.08-2.93 (m, 3H), 2.44-2.42 (m, 4H), 2.30-2.20 (m, 2H), 2.08 (s, 3H); ¹³C NMR (75 MHz, CDCl₃): δ 169.1, 156.4, 156.0, 145.7, 136.1, 135.0, 132.8, 129.8, 129.2, 128.1, 127.5, 125.2, 124.6, 62.7, 53.3, 52.9, 51.0, 46.4, 41.5, 35.3, 28.7, 21.5.

Preparation of (S)-3-{6-[(4-acetylpiperazin-1-yl)methyl]-1,2,3,4-tetrahydro-naphthalen-2-yl}-6-(4-fluorophenyl)thieno[3,2-d]pyrimidin-4(3H)-one (7): A suspension of 3-[6-(4-acetylpiperazin-1-ylmethyl)-1,2,3,4-tetrahydronaphthalen-2-yl]-6-bromothieno[3,2-d]pyrimidin-4(3H)-one, 6, (75 mg, 0.15 mmol), 4-flourobenzeneboronic acid (31 mg, 0.225 mmol, 1.5 eq), tetrakis(triphenylphosphine)palladium(0) (5 mol %), and potassium carbonate (50 nig, 0.38 mmol, 2.5 eq.) in water (1 mL) and dioxane (1 mL) is heated in the CEM microwave at 120° C. for 30 min. The suspension is cooled and partitioned between dichloromethane (10 mL) and water (5 mL). The layers are separated and the aqueous layer extracted with dichloromethane (2×10 mL). The organic layers are combined, washed with brine, dried over magnesium sulfate, filtered, and the filtrate concentrated to dryness. The crude material is purified over (3% MeOH/CH₂Cl₂) to afford the desired product. ¹H NMR (300 MHz, CDCl₃): δ 8.05 (s, 1H), 7.63-7.59 (m, 2H), 7.32 (s, 1H), 7.10-6.98 (m, 5H), 5.23-5.18 (m, 1H), 3.57-3.54 (m, 2H), 3.41 (bs, 4H), 3.25 (dd, J=16.1, 5.3 Hz, 1H), 3.04-2.89 (m, 3H), 2.34-2.35 (m, 4H), 2.24-2.18 (m, 2H), 2.01 (s, 3H); ¹³C NMR (75 MHz, CDCl₃): δ 169.1, 163.6 (d, J=250 Hz), 157.1, 156.9, 151.7, 145.5, 136.1, 135.1, 132.9, 129.7, 129.5, 129.2, 128.5, (d, J=8 Hz), 127.4, 122.8, 120.4, 116.5 (d, J=22 Hz), 62.7, 53.2, 52.9, 50.8, 46.4, 41.5, 35.4, 28.8, 28.7, 21.5; HRMS calcd for C₂₉H₃₀N₄O₂SF (M+H⁺): 517.2074, found 517.2074.

The following are non-limiting examples of other examples of R¹ units according to the present invention.

3-{(S)-6-[(4-Acetylpiperazin-1-yl)methyl]-1,2,3,4-tetrahydronaphthalen-2-yl}-6-(2-fluorophenyl)thieno[3,2-d]pyrimidin-4(3H)-one: ¹H NMR (300 MHz, CDCl₃): δ 8.15 (s, 1H), 7.75-7.70 (m, 1H), 7.68 (s, 1H), 7.44-7.37 (m, 1H), 7.28-7.07 (series of m, 5H), 5.36-5.26 (m, 1H), 3.71-3.63 (m, 2H), 3.50-3.47 (m, 4H), 3.34 (dd, J=16.2, 5.4 Hz, 1H), 3.13-3.02 (m, 3H), 2.45-2.44 (m, 4H), 2.35-2.26 (m, 2H), 2.10 (s, 3H); ¹³C NMR (75 MHz, CDCl₃): δ 169.1, 159.7 (d, J=253 Hz), 157.1, 156.7, 145.8, 145.4, 136.1, 132.9, 131.1 (d, J=8 Hz), 129.7, 129.2, 127.4, 125.0 (d, J=3 Hz), 123.9 (d, J=7 Hz), 123.3, 121.2 (d, J=12.2 Hz), 116.9 (d, J=22 Hz), 62.7, 53.2, 52.9, 50.8, 46.4, 41.5, 35.4, 28.8, 28.7, 21.5; HRMS calcd for C₂₉H₃₀N₄O₂SF (M+H⁺): 517.2074, found 517.2076.

3-{(S)-6-[(4-Acetylpiperazin-1-yl)methyl]-1,2,3,4-tetrahydronaphthalen-2-yl}-6-(3-fluorophenyl)thieno[3,2-d]pyrimidin-4(3H)-one: ¹H NMR (300 MHz, CDCl₃): δ 8.12 (s, 1H), 7.49-7.37 (series of m, 4H), 7.12-7.04 (series of m, 4H), 5.31-5.24 (m, 1H), 3.62-3.60 (m, 2H), 3.47 (s, 4H), 3.32 (dd, J=16.2, 5.2 Hz, 1H), 3.09-3.01 (m, 3H), 2.42-2.41 (m, 4H), 2.30-2.23 (m, 2H), 2.07 (s, 3H); ¹³C NMR (75 MHz, CDCl₃): δ 169.1, 163.2 (d, J=248 Hz), 157.0, 151.2, 145.5, 136.1, 135.3 (d, J=8.1 Hz), 135.1, 132.8, 131.0 (d, J=8.1 Hz), 135.1, 132.8, 131.0 (d, J=8.4 Hz), 129.7, 129.2, 127.4, 123.3, 122.4, 121.3, 116.6 (d, J=21 Hz), 113.5 (d, J=23 Hz), 62.7, 53.2, 52.9, 50.8, 46.4, 41.5, 35.4, 28.7, 28.6, 21.5; HRMS calcd for C₂₉H₃₀N₄O₂SF(M+H⁺): 517.2074, found 517.2084.

(S)-3-{6-[(4-Acetylpiperazin-1-yl)methyl]-1,2,3,4-tetrahydronaphthalen-2-yl}-6-(3,4-difluorophenyl)thieno[3,2-d]pyrimidin-4(3H)-one (TFA salt): ¹H NMR (400 MHz, CD₃OD) δ 2.11 (s, 3H), 2.23-2.43 (m, 2H), 3.06-3.35 (m, 14H), 5.06-5.10 (m, 1H), 7.25-7.39 (m, 4H), 7.56-7.60 (m, 2H), 7.70-7.75 (m, 1H), 8.40 (s, 1H); ¹³C NMR (100 MHz, CD₃OD) δ 22.84, 28.73, 31.77, 35.38, 41.62, 46.52, 50.94, 52.92, 53.30, 87.62, 120.82, 123.12, 127.79, 129.58, 131.80, 135.70, 145.34, 151.31, 156.81; MS (LC/MS, ESI/pos): m/z 535 (M+H)⁺.

(S)-3-{6-[(4-Acetylpiperazin-1-yl)methyl]-1,2,3,4-tetrahydronaphthalen-2-yl}-6-(3,5-difluorophenyl)thieno[3,2-d]pyrimidin-4(3H)-one (TFA salt): ¹H NMR (400 MHz, CD₃OD) δ 2.15 (s, 3H), 2.23-2.43 (m, 2H), 3.06-3.35 (m, 14H), 5.06-5.10 (m, 1H), 7.00-7.05 (m, 1H), 7.22-7.41 (m, 5H), 7.67 (s, 1H), 8.40 (s, 1H); ¹³C NMR (100 MHz, CD₃OD) δ 22.84, 28.73, 31.77, 35.38, 41.62, 46.52, 50.94, 52.92, 53.30, 62.74, 121.59, 123.58, 125.77, 127.45, 128.30, 129.24, 129.72, 131.35, 132.79, 133.25, 133.74, 133.87, 135.08, 136.25, 145.69, 149.85; MS (LC/MS, ESI/pos): m/z 535 (M+H)⁺.

(S)-6-(3,4-Difluorophenyl)-3-{6-[(4-(methylsulfonyl)piperazin-1-yl]methyl}-1,2,3,4-tetrahydronaphthalen-2-yl)thieno[3,2-d]pyrimidin-4(3H)-one (TFA salt): ¹H NMR (400 MHz, DMSO-d6) δ 2.15-2.42 (m, 2H), 2.99-3.35 (m, 17H), 5.02-5.05 (m, 1H), 7.25-7.39 (m, 4H), 7.56-7.60 (m, 2H), 7.70-7.75 (m, 1H), 8.40 (s, 1H); ¹³C NMR (1400 MHz, DMSO-d6) δ 27.98, 29.38, 34.85, 51.01, 51.67, 103.50, 116.11, 116.30, 119.13, 119.31, 122.64, 122.85, 124.02, 130.07, 136.30, 149.32, 152.00, 156.80, 157.65, 158.82; MS (LC/MS, ESI/pos): m/z 571 (M+H)⁺.

(S)-3-{6-[(4-Acetylpiperazin-1-yl)methyl]-1,2,3,4-tetrahydronaphthalen-2-yl}-6-(2,4-difluorophenyl)thieno[3,2-d]pyrimidin-4(3H)-one (TFA salt): MS (LC/MS, ESI/pos): m/z 535 (M+H)⁺.

(S)-6-(2,4-Difluorophenyl)-3-{6-[(4-(methylsulfonyl)piperazin-1-yl]methyl}-1,2,3,4-tetrahydro-naphthalen-2-yl)thieno[3,2-d]pyrimidin-4(3H)-one (TFA salt): ¹H NMR (400 MHz, DMSO-d6) δ 2.15-2.42 (m, 2H), 2.99-3.35 (m, 17H), 5.02-5.05 (m, 1H), 7.24-7.36 (m, 4H), 7.51-7.56 (m, 1H), 7.87 (s, 1H), 8.04-8.12 (m, 1H), 8.59 (s, 1H); MS (LC/MS, ESI/pos): m/z 571 (M+H)⁺.

(S)-6-(3,5-Difluorophenyl)-3-(6-{[4-(methylsulfonyl)piperazin-1-yl]methyl}-1,2,3,4-tetrahydronaphthalen-2-yl)thieno[3,2-d]pyrimidin-4(3H)-one(TFA salt): ¹H NMR (400 MHz, DMSO-d6) δ 2.15-2.42 (m, 2H), 2.99-3.35 (m, 17H), 5.02-5.05 (m, 1H), 7.00-7.05 (m, 1H), 7.22-7.41 (m, 5H), 7.67 (s, 1H), 8.40 (s, 1H); ¹³C NMR (400 MHz, DMSO-d6) δ 27.96, 29.37, 34.87, 35.62, 43.31, 50.94, 51.71, 59.42, 105.53, 110.10, 123.12, 123.83, 129.13, 130.08, 136.32, 148.15, 148.83, 156.83, 157.43, 158.70, 159.01, 162.24, 164.83; MS (LC/MS, ESI/pos): m/z 571 (M+H)⁺.

3-{(S)-6-[(4-Acetylpiperazin-1-yl)methyl]-1,2,3,4-tetrahydronaphthalen-2-yl}-6-(3-chlorophenyl)thieno[3,2-d]pyrimidin-4(3H)-one: ¹H NMR (300 MHz, CDCl₃): δ 8.12 (s, 1H), 7.67 (s, 1H), 7.59-7.56 (m, 1H), 7.49 (s, 1H), 7.41-7.37 (m, 2H), 7.12-7.04 (m, 3H), 5.32-5.24 (m, 1H), 3.64-3.61 (m, 2H), 3.47-3.45 (m, 4H), 3.31 (dd, J=16.2, 5.4 Hz, 1H), 3.10-3.01 (m, 3H), 2.43-2.40 (m, 4H), 2.34-2.23 (m, 2H), 2.07 (s, 3H); ¹³C NMR (75 MHz, CDCl₃): δ 169.1, 157.0, 151.0, 145.5, 136.1, 135.4, 135.1, 134.9, 132.8, 130.6, 129.7, 129.6, 129.2, 127.4, 126.6, 124.8, 123.3, 121.3, 62.7, 53.2, 52.9, 50.8, 46.4, 41.5, 35.4, 28.7, 21.5; HRMS calcd for C₂₉H₃₀N₄O₂SCl (M+H⁺): 533.1778, found 533.1801.

(S)-3-{6-[(4-Acetylpiperazin-1-yl)methyl]-1,2,3,4-tetrahydronaphthalen-2-yl}-6-(2,4-dichlorophenyl)thieno[3,2-d]pyrimidin-4(3H)-one (TFA salt) (8j): MS(LC/MS/pos): 567.1(M+H)⁺.

(S)-6-(2,4-Dichlorophenyl)-3-{6-[(4-(methylsulfonyl)piperazin-1-yl]methyl}-1,2,3,4-tetrahydronaphthalen-2-yl)thieno[3,2-d]pyrimidin-4(3H)-one (TFA salt): ¹H NMR (400 MHz, DMSO-d6) δ 9.92 (bs, 1H), 8.60 (s, 1H), 7.88-7.87 (m, 1H), 7.81-7.79 (m, 1H), 7.75 (s, 1H), 7.61-7.59 (m, 1H), 7.31-7.23 (m, 3H), 5.07-5.00 (m, 1H), 4.34 (bs, 2H), 3.76-3.03 (m, 12H), 3.01 (s, 3H), 2.45-2.38 (m, 1H), 2.18-2.14 (m, 1H). MS (M+1): 603.1.

(S)-3-{6-[(4-Acetylpiperazin-1-yl)methyl]-1,2,3,4-tetrahydronaphthalen-2-yl}-6-(3,4-dichlorophenyl)thieno[3,2-d]pyrimidin-4(3H)-one (TFA salt): ¹H NMR (400 MHz, CDCl₃) δ 0.85-0.90 (m, 3H), 1.25-1.30 (m, 3H), 2.09 (s, 3H), 2.24-2.27 (m, 2H), 3.00-3.65 (m, 8H), 5.28-5.30 (m, 1H), 7.06-7.13 (m, 3H), 7.49-7.53 (m, 3H), 7.78 (s, 1H), 8.14 (s, 1H); ¹³C NMR (100 MHz, CDCl₃) δ 22.84, 28.73, 31.77, 35.38, 41.62, 46.52, 50.94, 52.92, 53.30, 62.74, 121.59, 123.58, 125.77, 127.45, 128.30, 129.24, 129.72, 131.35, 132.79, 133.25, 133.74, 133.87, 135.08, 136.25, 145.69, 149.85, 156.95, 169.13; MS (LC/MS, ESI/pos): m/z 568 (M+H)⁺.

(S)-6-(3,4-Dichlorophenyl)-3-{6-[(4-(methylsulfonyl)piperazin-1-yl]methyl}-1,2,3,4-tetrahydronaphthalen-2-yl)thieno[3,2-d]pyrimidin-4(3H)-one (TFA salt): ¹H NMR (400 MHz, DMSO-d6) δ 2.15-2.42 (m, 2H), 2.99-3.35 (m, 17H), 5.02-5.05 (m, 1H), 7.06-7.13 (m, 3H), 7.49-7.53 (m, 3H), 7.78 (s, 1H), 8.14 (s, 1H); ¹³C NMR (100 MHz, DMSO-d6) δ 19.12, 27.45, 28.80, 34.43, 38.21, 42.95, 51.05, 51.17, 52.18, 53.10, 60.19, 116.55, 117.45, 122.15, 122.85, 126.72, 128.75, 131.62, 136.63, 137.00, 146.92, 155.92, 159.27, 169.98; MS (LC/MS, ESI/pos): m/z 604 (M+H)⁺.

(S)-4-(3-{6-[(4-Acetylpiperazin-1-yl)methyl]-1,2,3,4-tetrahydronaphthalen-2-yl}-4-oxo-3,4-dihydrothieno[3,2-d]pyrimidin-6-yl)benzonitrile (TFA salt): ¹H NMR (CD₃OD): δ 2.15 (s, 3H), 2.29-2.44 (m, 2H), 3.10-3.27 (m, 6H), 3.79-3.87 (m, 3H), 4.26-4.29 (s, 2H), 4.73 (bs, 3H), 5.20-5.22 (m, 1H), 7.23-7.30 (m, 3H), 7.58-7.60 (bs, 1H), 7.71 (s, 1H), 7.81-7.83 (m, 2H), 7.91-7.94 (m, 2H), 8.32 (s, 1H); MS(LC/MS/pos): 524.0 (M+H)⁺.

(S)-3-(3-{6-[(4-(Methylsulfonyl)piperazin-1-yl]methyl}-1,2,3,4-tetrahydro-naphthalen-2-yl)-4-oxo-3,4-dihydrothieno[3,2-d]pyrimidin-6-yl)benzonitrile (TFA salt): ¹H NMR (400 MHz, DMSO-d6) δ 9.94 (bs, 1H), 8.58 (s, 1H), 8.43 (s, 1H), 8.20-8.18 (m, 1H), 8.07 (s, 1H), 7.94-7.92 (m, 1H), 7.72 (t, J=7.80 Hz, 1H), 7.29-7.22 (m, 3H), 5.07-4.99 (m, 3H), 4.33 (bs, 1H), 3.45-3.01 (m, 15H), 3.00 (s, 1H), 2.46-2.37 (m, 1H), 2.17-2.15 (m, 1H). MS (M+1): 560.1.

(S)-3-(3-{6-[(4-Acetylpiperazin-1-yl)methyl]-1,2,3,4-tetrahydronaphthalen-2-yl}-4-oxo-3,4-dihydrothieno[3,2-d]pyrimidin-6-yl)benzonitrile (TFA salt): ¹H NMR (CD₃OD): δ 2.11 (s, 3H), 2.21-2.25 (m, 1H), 2.40-2.45 (m, 1H), 3.07-3.08 (m, 3H), 4.30 (s, 2H), 4.81 (s, 9H), 5.06-5.12 (m, 1H), 7.25-7.30 (m, 3H), 7.62-7.66 (bt, 1H), 7.73-7.77 (m, 2H), 8.05-8.07 (m, 1H), 8.16 (bs, 1H), 8.40 (s, 1H); MS (LC/MS/pos): 524.1 (M+H)⁺.

(S)-3-{6-[(4-Acetylpiperazin-1-yl)methyl]-1,2,3,4-tetrahydronaphthalen-2-yl}-6-p-tolylthieno[3,2-d]pyrimidin-4(3H)-one: ¹H NMR (300 MHz, DMSO d₆): δ 8.58 (s, 1H), 7.83 (s, 1H), 7.77 (d, J=8.1 Hz, 2H), 7.42-7.40 (m, 2H), 7.32 (d, J=8.1 Hz, 2H), 7.21 (d, J=8.2 Hz, 1H), 5.02-4.85 (m, 1H), 4.44-4.36 (m, 1H), 4.27 (s, 2H), 4.01-3.96 (m, 1H), 3.63-3.55 (m, 1H), 3.31-2.85 (series of m, 10H), 2.36 (s, 3H), 2.16-2.13 (m, 1H), 2.03 (s, 3H); ¹³C NMR (300 MHz, DMSO d₆): δ 168.6, 157.1, 156.1, 151.5, 147.3, 139.7, 136.0, 135.5, 131.8, 129.9, 129.7, 129.4, 128.9, 127.2, 126.1, 121.0, 120.3, 58.4, 50.9, 50.5, 50.1, 42.3, 37.6, 34.3, 28.8, 27.4, 21.0, 20.9; HRMS calcd for C₃₀H₃₃N₄O₂S(M+H⁺): 513.2324, found 513.2333.

(S)-3-{6-[(4-Acetylpiperazin-1-yl)methyl)-1,2,3,4-tetrahydronaphthalen-2-yl)-6-(pyridin-3-yl)thieno[3,2-d]pyrimidin-4(3H)-one (TFA salt): ¹H NMR (400 MHz, DMSO-d6) δ 10.11 (bs, 1H), 9.14-9.13 (m, 1H), 8.68-8.66 (m, 1H), 8.59 (s, 1H), 8.33-8.30 (m, 1H), 8.05 (s, 1H), 7.60-7.56 (m, 1H), 7.31-7.23 (m, 3H), 5.07-5.00 (m, 1H), 4.31 (s, 3H), 3.99 (bs, 1H), 3.29-2.91 (m, 10H), 2.47-2.38 (m, 1H), 2.18-2.15 (m, 1H), 2.04 (s, 3H). MS (M+1): 500.1.

(S)-3-{6-[(4-(Methylsulfonyl)piperazin-1-yl]methyl}-1,2,3,4-tetrahydro-naphthalen-2-yl)-6-(pyridin-3-yl)thieno[3,2-d]pyrimidin-4(3H)-one (TFA salt): ¹H NMR (400 MHz, DMSO-d6) δ 10.12 (bs, 1H), 9.09-9.08 (m, 1H), 8.62-8.61 (m, 1H), 8.53 (s, 1H), 8.29-8.26 (m, 1H), 7.99 (s, 1H), 7.55-7.52 (m, 1H), 7.26 (s, 1H), 7.19 (q, J=8.19 Hz, 2H), 5.01-4.94 (1H), 4.29 (s, 2H), 3.67-2.97 (m, 12H), 2.95 (s, 3H), 2.41-2.32 (m, 1H), 2.12-2.09 (m, 1H); ¹³C NMR (100 MHz, DMSO-d6) δ 157.59, 156.81, 150.43, 148.11, 147.96, 146.98, 136.88, 136.39, 134.90, 132.17, 130.14, 129.41, 129.33, 127.87, 125.10, 123.20, 115.02, 59.16, 51.67, 50.80(2), 43.05(2), 35.75, 34.88, 29.36, 27.94. MS (M+1): 536.1.

(S)-3-{6-[(4-Acetylpiperazin-1-yl)methyl]-1,2,3,4-tetrahydronaphthalen-2-yl}-6-(pyridin-4-yl)thieno[3,2-d]pyrimidin-4(3H)-one (TFA salt): ¹H NMR (CD₃OD) δ 2.10 (s, 3H), 2.21-2.26 (m, 2H), 2.37-2.48 (m, 1H), 3.03-3.09 (m, 3H), 3.27-3.37 (m, 6H), 4.29 (s, 2H), 4.87 (m, 4H), 5.05-5.13 (m, 1H), 7.22-7.30 (m, 3H), 8.17 (s, 1H), 8.33-8.34 (m, 2H), 7.46 (s, 1H), 8.82-8.84 (bd, 2H); MS (LC/MS/pos): 500.1 (M+H)⁺.

(S)-3-{6-[(4-(Methylsulfonyl)piperazin-1-yl]methyl}-1,2,3,4-tetrahydro-naphthalen-2-yl)-6-(pyridin-4-yl)thieno[3,2-d]pyrimidin-4(3H)-one (TFA salt): MS(LC/MS/pos): 537(M+H)⁺.

(S)-3-(6-((4-Acetylpiperazin-1-yl)methyl)-1,2,3,4-tetrahydronaphthalen-2-yl)-6-(2-fluoropyridin-3-yl)thieno[3,2-d]pyrimidin-4(3H)-one (TFA salt): ¹H NMR (CDCl₃): δ 2.12 (s, 3H), 2.28-2.34 (m, 3H), 3.00-3.17 (m, 5H), 3.29-3.38 (m, 2H), 3.84 (bs, 3H), 4.13-4.20 (m, 3H), 5.22-5.28 (m, 1H), 7.13-7.26 (m, 4H), 7.35 (t, 1H), 7.76 (s, 1H), 8.12-8.18 (m, 2H), 8.26-8.27 (d, 1H); MS(LC/MS/pos): 518.1 (M+H)⁺.

(S)-3-{6-[(4-Acetylpiperazin-1-yl)methyl]-1,2,3,4-tetrahydronaphthalen-2-yl}-6-(6-fluoropyridin-3-yl)thieno[3,2-d]pyrimidin-4(3H)-one (TFA salt): ¹H NMR (CD₃OD) δ 2.15 (s, 3H), 2.24-2.30 (m, 1H), 2.41-2.51 (m, 1H), 3.08-3.13 (m, 3H), 4.36 (s, 2H), 4.85 (s, 10H), 5.09-5.17 (m, 1H), 7.20-7.23 (m, 1H), 7.30-7.34 (m, 3H), 7.72 (s, 1H), 8.34-8.38 (m, 1H), 8.45 (s, 1H), 8.66-7.67 (m, 1H); MS(LC/MS/pos): 532.3 (M+H)⁺.

(S)-6-(6-Fluoropyridin-3-yl)-3-{6-[(4-(methylsulfonyl)piperazin-1-yl]methyl}-1,2,3,4-tetrahydronaphthalen-2-yl)thieno[3,2-d]pyrimidin-4(3H)-one (TFA salt): MS(LC/MS/pos): 554.1 (M+H)⁺.

(S)-3-{6-[(4-Acetylpiperazin-1-yl)methyl]-1,2,3,4-tetrahydronaphthalen-2-yl}-6-(6-chloropyridin-3-yl)thieno[3,2-d]pyrimidin-4(3H)-one (TFA salt): 1H NMR (400 MHz, DMSO-d6) δ 9.90 (bs, 1H), 8.98-8.97 (m, 1H), 8.59 (s, 1H), 8.36-8.33 (m, 1H), 8.08 (s, 1H), 7.69-7.67 (m, 1H), 7.30-7.23 (m, 3H), 5.06-4.95 (m, 1H), 4.44 (bs, 1H), 4.29 (bs, 2H), 4.02-3.98 (m, 1H), 3.37-2.88 (m, 10H), 2.46-2.37 (m, 1H), 2.19-2.15 (m, 1H), 2.04 (s, 3H). MS (M+1): 534.0.

(S)-3-{6-[(4-Acetylpiperazin-1-yl)methyl]-1,2,3,4-tetrahydronaphthalen-2-yl}-6-(2-chloropyridin-4-yl)thieno[3,2-d]pyrimidin-4(3H)-one (TFA salt): ¹H NMR (CDCl₃): δ 2.12 (s, 3H), 2.25-2.34 (m, 3H), 3.02-3.14 (m, 5H), 3.33-3.38 (m, 2H), 3.84 (bs, 3H), 4.12-4.19 (m, 3H), 5.21-5.27 (m, 1H), 7.14-7.19 (m, 2H), 7.23-7.26 (bd, 2H), 7.50-7.52 (m, 1H), 7.63 (bs, 1H), 7.70 (s, 1H), 8.17 (s, 1H), 7.49 (d, 1H); MS (LC/MS/pos): 534.1 (M+H)⁺.

(S)-6-(6-Chloropyridin-3-yl)-3-{6-[(4-(methylsulfonyl)piperazin-1-yl]methyl}-1,2,3,4-tetrahydronaphthalen-2-yl)thieno[3,2-d]pyrimidin-4(3H)-one (TFA salt): MS(LC/MS/pos): 570.1 (M+H)⁺.

(S)-6-(2-Chloropyridin-4-yl)-3-{6-[(4-(methylsulfonyl)piperazin-1-yl]methyl}-1,2,3,4-tetrahydronaphthalen-2-yl)thieno[3,2-d]pyrimidin-4(3H)-one (TFA salt): MS(LC/MS/pos): 570.1 (M+H)⁺.

(S)-3-{6-[(4-Acetylpiperazin-1-yl)methyl]-1,2,3,4-tetrahydronaphthalen-2-yl}-6-(2-methoxypyridin-3-yl)thieno[3,2-d]pyrimidin-4(3H)-one (TFA salt): ¹H NMR (400 MHz, CD₃OD) δ 2.14 (s, 3H), 2.23-2.43 (m, 2H), 3.06-3.35 (m, 14H), 4.10 (s, 3H), 5.06-5.10 (m, 1H), 7.09 (m, 1H), 7.27-7.34 (m, 3H), 7.82 (s, 1H), 8.20-8.23 (m, 2H), 8.42 (s, 1H); ¹³C NMR (100 MHz, CD₃OD) δ 19.65, 27.78, 28.80, 34.43, 38.21, 42.95, 51.05, 51.17, 52.18, 53.10, 60.19, 116.09, 117.45, 122.15, 122.85, 126.72, 128.75, 130.00, 131.62, 136.63, 137.00, 146.92, 155.92, 157.53, 159.82; 170.54; MS (LC/MS, ESI/pos): m/z 530 (M+H)⁺.

(S)-6-(6-Methoxypyridin-3-yl)-3-{6-[(4-(methylsulfonyl)piperazin-1-yl]methyl}-1,2,3,4-tetrahydronaphthalen-2-yl)thieno[3,2-d]pyrimidin-4(3H)-one (TFA salt): ¹H NMR (400 MHz, DMSO-d6) δ 9.87 (bs, 1H), 8.73-8.72 (m, 1H), 8.56 (s, 1H), 8.21-8.19 (m, 1H), 7.88 (s, 1H), 7.31-7.23 (m, 3H), 6.98-6.96 (m, 1H), 5.07-4.99 (m, 1H), 4.34 (bs, 2H), 3.92 (s, 3H), 3.50-3.03 (m, 12H), 3.01 (s, 3H), 2.45-2.37 (m, 1H), 2.18-2.13 (m, 1H). MS (M+1): 566.1.

(S)-3-{6-[(4-Acetylpiperazin-1-yl)methyl]-1,2,3,4-tetrahydronaphthalen-2-yl}-6-(6-methoxypyridin-3-yl)thieno[3,2-d]pyrimidin-4(3H)-one (TFA salt): ¹H NMR (400 MHz, CD₃OD) δ 2.13 (s, 3H), 2.23-2.43 (m, 2H), 3.06-3.35 (m, 17H), 5.06-5.10 (m, 1H), 7.12-7.23 (m, 2H), 7.30-7.34 (m, 4H), 7.88-7.94 (m, 1H), 8.43 (s, 1H); ¹³C NMR (100 MHz, CD₃OD) δ 19.64, 27.74, 28.79, 34.37, 38.20, 42.95, 51.19, 52.30, 60.22, 126.70, 128.75, 130.03, 131.62, 136.66, 137.04, 146.84, 156.56, 157.39, 170.54; MS (LC/MS, ESI/pos): m/z 530 (M+H)⁺.

(S)-6-(2-Methoxypyridin-3-yl)-3-{6-[(4-(methylsulfonyl)piperazin-1-yl]methyl}-1,2,3,4-tetrahydronaphthalen-2-yl)thieno[3,2-d]pyrimidin-4(3H)-one (TFA salt): ¹H NMR (DMSO-d6): δ 2.09-2.12 (m, 1H), 2.32-2.41 (m, 1H), 2.95-2.30 (m, 5H), 3.01-3.25 (m, 6H), 3.57-3.59 (bs, 2H), 4.03 (s, 5H), 4.28 (bs, 2H), 4.94-5.01 (m, 1H), 7.11-7.26 (m, 4H), 7.99 (s, 1H), 8.22 (m, 1H), 8.36 (m, 1H), 8.51 (s, 1H), 10.06 (bs, 1H); MS (LC/MS/pos): 566.1 (M+H)⁺.

(S)-3-{6-[(4-Acetylpiperazin-1-yl)methyl]-1,2,3,4-tetrahydronaphthalen-2-yl}-6-(thiophen-2-yl)thieno[3,2-d]pyrimidin-4(3H)-one: ¹HNMR (400 MHz, DMSO-d6) δ 10.06 (bs, 1H), 8.56 (s, 1H), 7.75-7.74 (m, 1H), 7.67-7.66 (m, 1H), 7.64 (s, 1H), 7.30-7.19 (m, 4H), 5.05-4.97 (m, 1H), 4.44(bs, 2H), 4.33 (bs, 2H), 4.03-3.94 (m, 1H), 3.35-2.87 (m, 10H), 2.47-2.36 (m, 1H), 2.17-2.13 (m, 1H), 2.04 (s, 3H); 13C NMR (100 MHz, DMSO-d6) δ 169.18, 157.59, 155.64, 148.14, 145.08, 136.87, 136.36, 135.78, 132.20, 130.11, 129.48, 129.22, 127.73(2), 121.32, 121.08, 59.40, 51.61, 51.36, 51.01, 43.17, 38.39, 34.90, 29.36, 27.94, 21.58. MS (M+1): 505.1.

(S)-3-{6-[(4-Acetylpiperazin-1-yl)methyl]-1,2,3,4-tetrahydronaphthalen-2-yl}-6-(thiophen-3-yl)thieno[3,2-d]pyrimidin-4(3H)-one (TFA salt): ¹H NMR (400 MHz, DMSO-d6) δ 9.89 (bs, 1H), 8.55 (s, 1H), 8.14-8.13 (m, 1H), 7.77 (s, 1H), 7.75-7.73 (m, 1H), 7.66-7.64 (m, 1H), 7.30-7.23 (m, 3H), 5.05-4.97 (m, 1H), 4.49-4.42 (m, 1H), 4.29 (bs, 3H), 4.05-3.96 (m, 1H), 3.38-2.86 (m, 9H), 2.45-2.36 (m, 1H), 2.17-2.13 (m, 1H), 2.04 (s, 3H). MS (M+1): 505.1.

(S)-3-{6-[(4-(Methylsulfonyl)piperazin-1-yl]methyl}-1,2,3,4-tetrahydro-naphthalen-2-yl)-6-(thiophen-3-yl)thieno[3,2-d]pyrimidin-4(3H)-one (TFA salt): ¹H NMR (400 MHz, CDCl₃) δ 8.21 (s, 1H), 7.66-7.65 (m, 1H), 7.45-7.43 (m, 1H), 7.42-7.40 (m, 3H), 7.18 (s, 3H), 5.30-5.22 (m, 1H), 4.15 (s, 2H), 3.89-3.48 (bs, 6H), 3.39-3.33 (s, 2H), 3.13-3.06 (m, 4H), 2.88 (s, 3H), 2.36-2.27 (m, 2H); ¹³C NMR (100 MHz, CDCl3) δ 156.72, 155.91, 148.20, 145.49, 136.79, 136.76, 134.55, 131.77, 130.40, 128.99, 127.82, 127.59, 126.10, 125.88, 123.39, 119.58, 61.17, 51.41(2), 50.95, 42.29(2), 36.33, 35.49, 28.81, 28.34. MS (M+1): 541.1.

(S)-3-{6-([4-(Methylsulfonyl)piperazin-1-yl]methyl}-1,2,3,4-tetrahydro-naphthalen-2-yl)-6-(thiophen-2-yl)thieno[3,2-d]pyrimidin-4(3H)-one (TFA salt): ¹H NMR (400 MHz, DMSO-d6) δ 9.84 (bs, 1H), 8.55 (s, 1H), 7.75-7.73 (m, 1H), 7.67-7.66 (m, 1H), 7.64 (s, 1H), 7.29-7.19 (m, 4H), 5.06-4.99 (m, 1H), 4.33 (bs, 1H), 3.73-3.02 (m, 13H), 3.00 (s, 3H), 2.46-2.36 (m, 1H), 2.18-2.13 (m, 1H). MS (M+1): 541.1.

(S)-3-{6-[(4-Acetylpiperazin-1-yl)methyl]-1,2,3,4-tetrahydronaphthalen-2-yl}-6-(furan-3-yl)thieno[3,2-d]pyrimidin-4(3H)-one (TFA salt): ¹H NMR (400 MHz, CD₃OD) δ 2.15 (s, 3H), 2.23-2.43 (m, 2H), 3.06-3.35 (m, 14H), 5.06-5.10 (m, 1H), 6.86 (s, 1H), 7.29-7.42 (m, 4H), 7.65 (s, 1H), 8.11 (s, 1H), 8.41 (s, 1H); ¹³C NMR (100 MHz, CD₃OD) δ 19.89, 27.78, 28.80, 34.43, 38.13, 42.95, 51.17, 52.18, 53.10, 60.19, 116.09, 117.45, 122.85, 126.72, 128.75, 130.00, 131.45, 136.63, 137.00, 146.92, 155.92, 157.78, 159.82, 170.66; MS (LC/MS, ESI/pos): m/z 489 (M+H)⁺.

(S)-6-(Furan-3-yl)-3-{6-[(4-(methylsulfonyl)piperazin-1-yl]methyl}-1,2,3,4-tetrahydronaphthalen-2-yl)thieno[3,2-d]pyrimidin-4(3H)-one (TFA salt): ¹H NMR (400 MHz, DMSO-d6) δ 8.49 (s, 1H), 8.37 (s, 1H), 7.79 (s, 1H), 7.60 (s, 1H), 7.01 (m, 4H), 4.93 (m, 1H), 3.41 (s, 2H), 3.23 (s, 2H), 2.80-3.18 (m, 8H), 2.40 (m, 5H), 2.07 (m, 2H). ¹³C NMR (100 MHz, DMSO-d6) δ 157.6, 156.7, 147.9, 145.8, 143.3, 141.8, 136.4, 135.5, 133.9, 129.8, 129.5, 127.3, 121.5, 120.9, 120.5, 109.8, 62.0, 52.4, 51.6, 46.1, 35.0, 34.3, 29.5, 28.3. MS (M+H): 525.1

Compounds listed and described herein above have been found in many instances to exhibit activities (IC₅₀ in the cell based assay described herein below or ones which are referenced herein) at a level below 1 micromolar (μM).

Each of the disease states or conditions which the formulator desires to treat may require differing levels or amounts of the compounds described herein to obtain a therapeutic level. The formulator can determine this amount by any of the common testing procedures known to the artisan.

The present invention further relates to forms of the present compounds, which under normal human or higher mammalian physiological conditions, release the compounds described herein. One iteration of this aspect includes the pharmaceutically acceptable salts of the analogs described herein. The formulator, for the purposes of compatibility with delivery mode, excipients, and the like, can select if necessary one salt form of the present analogs over another since the compounds themselves are the active species which mitigate the disease processes described herein.

Pro-Drug Forms

Related to this aspect are the various precursor or “pro-drug” forms of the analogs of the present invention. It may be desirable to formulate the compounds of the present invention as a chemical species which itself is not an antagonist against melanin concentrating hormone as described herein, but instead are forms of the present analogs which when delivered to the body of a human or higher mammal will undergo a chemical reaction catalyzed by the normal function of the body, inter alia, enzymes present in the stomach, blood serum, said chemical reaction releasing the parent analog. The term “pro-drug” relates to these species which are converted in vivo to the active pharmaceutical.

The pro-drugs of the present invention can have any form suitable to the formulator, for example, esters are common pro-drug forms. In the present case, however, the pro-drug may necessarily exist in a form wherein a covalent bond is cleaved by the action of an enzyme present at the target situs. For example, a C—C covalent bond may be selectively cleaved by one or more enzymes at said target situs and, therefore, a pro-drug in a form other than an easily hydrolysable precursor, inter alia, esters, amides, and the like, may be utilized.

For the purposes of the present invention the term “therapeutically suitable pro-drug” is defined herein as “a melanin concentrating hormone antagonist modified in such a way as to be transformed in vivo to the therapeutically active form, whether by way of a single or by multiple biological transformations, when in contact with the tissues of humans or mammals to which the pro-drug has been administered, and without undue toxicity, irritation, or allergic response, and achieving the intended therapeutic outcome.”

A detailed description of pro-drug derivatives can be found in the following included herein by reference:
a) Design of Produrgs, edited by H. Bundgaard, (Elsevier, 1985);
b) Methods in Enzymology, 42, 309-396, edited by K. Widder et al. (Academic Press, 1985);
c) A Textbook of Drug Design and Development, edited by Krogsgaard-Larsen and H. Bundgaard, Chapter 5, “Design and Application of Prodrugs.” By H. Bundgaard, 113-191 (1991);
d) Advance Drug Delivery Reviews, H. Bundgaard, 8, 1-38 (1992);
e) Chem Pharm Bull, N. Kakeya et al., 32, 692 (1984).

### Formulations

The present invention also relates to compositions or formulations which comprise the melanin concentrating hormone antagonists according to the present invention. In general, the second aspect of the present invention relates to pharmaceutical compositions said compositions comprising:
A) An effective amount of one or more of the melanin concentrating hormone antagonists described herein; and
B) One or more pharmaceutically acceptable excipients.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

### Method of Use

Many human and mammalian disorders result from too much body mass (obesity or other over weight condition). Controlling body mass is a first step in preventing, as well as effectively treating many diseases and disease states. Among the disorders which are modulated, attenuated, abated, or otherwise controlled by the compounds of the present invention which serve as antagonists of MCH activity, is human obesity. This condition has been shown to be directly related to a wide range of disorders. The compounds of the selective antagonists of the present invention are capable of treating diseases acting as antagonists of MCH activity with minimal, little, or no activity involving the 5-HT_{2c} receptor.

As antagonists of MCH action upon the MCH receptor, the compounds of the present invention are useful in treating disorders that are mediated by MCH through the MCH receptor. Additional disorders other than obesity and food intake related illnesses that are mediated by MCH through the MCH receptor are abnormalities in reproduction and sexual behavior (sexual dysfunction, penile erection), thyroid hormone secretion, diuresis and water/electrolyte homeostasis, sensory processing, memory, sleep and arousal, anxiety and depression, seizure and in treatment of neurodegeneration or psychiatric disorders. In addition, melanin concentrating hormone antagonists are also effective in treating disorders relating to cardiovascular function, inflammation, sepsis, cardiogenic and hypovolemic shock, muscle atrophy, nerve growth and repair, intrauterine fetal growth, and the like.

The compounds of the present invention have improved cellular potency and pharmacokinetic properties and this advantage is made use of by the fact the third aspect of the present invention as a whole, relates to a method for controlling obesity, and the subsequent weight management after weight loss. This is achieved by administering to a human or a higher mammal an effective amount of one or more of the compounds (analogs) as described herein. Non-limiting examples of diseases which are affected by an MCH antagonist activity are obesity and other body weight disorders, inter alia, anorexia and cachexia.

Melanin Concentrating Hormone (MCH) activity, to which the antagonists of the present invention are directed, and as discussed herein above, is not limited to modulation of food intake as effects on the hypothalamic-pituitary axis have been reported.²

The role of MCH in modulating a variety of biological functions, and, therefore, multiple disease states, was first established by Hawes et al. when they showed “that the MCH receptor couples to multiple G proteins to mediate several diverse intracellular signaling pathways.”³

MCH is expressed in the lateral hypothalamic area, which also has an important role in the regulation of the autonomic nervous system, heart rate, and blood pressure. Astrand et al., showed that male mice lacking the rodent MCH receptor demonstrated a significantly increased heart rate with no significant difference in mean arterial pressure.⁴

Utilizing the melanin concentrating hormone antagonists of the present invention will therefore affect a variety of diseases, disease states, conditions, or syndromes resulting from body weight disorders, inter alia, insulin resistance, glucose intolerance, Type-2 diabetes mellitus, coronary artery disease, elevated blood pressure, hypertension, dyslipidaemia, cancer (e.g., endometrial, cervical, ovarian, breast, prostate, gallbladder, colon), menstrual irregularities, hirsutism, infertility, gallbladder disease, restrictive lung disease, sleep apnea, gout, osteoarthritis, and thromboembolic disease.
2. Critical Rev. in Neurobiol., Nahon, 8, 221-262 (1994).
3. “The melanin-concentrating hormone receptor couples to multiple G proteins to activate diverse intracellular signaling pathways,” Hawes, B. E. et al., Endocrinology, 141(12), 4524-32 (2000).
4. “Mice lacking the Melanin Concentrating Hormone Receptor 1 demonstrate increased heart associated with altered autonomic activity,” Astand, A. et al., Am J Physiol Regul Integr Comp Physiol. May 6, (2004)

Although the melanin concentrating hormone antagonists of the present invention are discrete chemical entities, the method of delivery or the method of use may be coupled with other suitable drug delivery systems. For example, a drug delivery technique useful for the compounds of the present invention is the conjugation of the compound to an active molecule capable of being transported through a biological barrier.⁵ A specific example constitutes the coupling of the compound of the invention to fragments of insulin to achieve transport across the blood brain barrier.⁶
5. Zlokovic, B. V., Pharmaceutical Research, Vol. 12, pp. 1395-1406 (1995).
6. Fukuta, M., et al. Pharmaceutical Res., Vol. 11, pp. 1681-1688 (1994).
   For general reviews of technologies for drug delivery suitable for the compounds of the invention see:

Zlokovic, B. V., Pharmaceutical Res., Vol. 12, pp. 1395-1406 (1995) and Pardridge, W M, Pharmacol. Toxicol., Vol. 71, pp. 3-10 (1992).

The compounds of the present invention which are selective antagonists at the MCH-R1 receptor over the 5-HT_{2c} receptor are suitable for use the following:

A method for controlling the body weight of humans and higher mammals, said method comprising administering to a human or higher mammal an effective amount of one or more selective antagonist of the present invention, including all enantiomeric and diastereomeric forms and pharmaceutically acceptable salts thereof.

A method for controlling weight loss in humans and higher mammals, said method comprising administering to a human or higher mammal an effective amount of one or more selective antagonist of the present invention, including all enantiomeric and diastereomeric forms and pharmaceutically acceptable salts thereof.

A method for controlling in humans one or more diseases, disease states, conditions, or syndromes relating to behavior, said diseases, disease states, conditions, or syndromes are chosen from memory impairment (including learning), cardiovascular function, inflammation, sepsis, cardiogenic and hypovolemic shock, sexual dysfunction, penile erection, muscle atrophy, nerve growth and repair, and intrauterine fetal growth comprising administering an effective amount of one or more selective antagonist of the present invention, including all enantiomeric and diastereomeric forms and pharmaceutically acceptable salts thereof.

A method for controlling in humans one or more diseases, disease states, conditions, or syndromes resulting from body weight disorders, said diseases, disease states, conditions, or syndromes are chosen from insulin resistance, glucose intolerance, Type-2 diabetes mellitus, coronary artery disease, elevated blood pressure, hypertension, dyslipidaemia, endometrial cancer, cervical cancer, ovarian cancer, breast cancer, prostate cancer, gallbladder cancer, colon cancer, menstrual irregularities, hirsutism, infertility, gallbladder disease, restrictive lung disease, sleep apnea, gout, osteoarthritis, and thromboembolic disease, said method comprising administering to a human an effective amount of one or more selective antagonist of the present invention, including all enantiomeric and diastereomeric forms and pharmaceutically acceptable salts thereof.

The compounds of the present invention are suitable for use as a medicament. The compounds of the present invention are also suitable for controlling obesity in humans and higher mammals. The compounds of the present invention are also suitable for use in the manufacture of a medicament, preferably a medicament for use in the treatment of any of the methods of treatment described above.

### Procedures

### Binding and Functional Assays for Melanin Concentrating Hormone (MCH)

In vitro binding and function assays are performed on membranes derived from cells or tissues expressing endogenous MCH1R. Competition binding assays are performed to identify high affinity compounds. Briefly, either radiolabeled or europium labeled MCH with varying concentrations of competitor compound which are incubated with membranes expressing the receptor. Rat brain membrane or cell lines, including but not limited to human Kelly neuroblastoma cells, A-431 epidermoid cells, and rat PC-12 cells are known to express endogenous MCH1R and are used in the assay. Binding is allowed to proceed until equilibrium is reached then bound labeled MCH is separated from free MCH by capturing membranes onto a filter. The filters are washed to remove loosely associated MCH and labeled MCH is quantified. Data is analyzed and IC₅₀ and Kᵢ are calculated to determine compound affinity.

MCH function assays are performed in an analogous manner to the binding assay. Competition assays are performed with a single concentration of MCH and varying concentrations of compound. Function is assayed using GTP binding or a functional response (e.g. Calcium uptake, MAP/ERK activation) because the MCH1R is a G-protein coupled receptor that couples the the G_{i/o} and G_{q} proteins and has been shown to elicit these cellular functional responses. The assay can be performed on the same membranes as used for the binding assays. There are readily available kits for measuring GTP binding to membranes (e.g. Perkin Elmer Life Sciences). Data is analyzed and IC₅₀ values are generated to determine whether the compound is an agonist or antagonist.

### Binding Assays for Serotonin Receptor, 5-HT_{2c} Receptor

MCH antagonist compounds are evaluated for binding to the serotonin 5-HT_{2c} receptor to determine receptor selectivity. Binding activity is assessed using a competitive assay with ³H-mesulergine (Perkin Elmer), a 5-HT_{2c} selective ligand, on membrane containing the 5-HT_{2c} receptor. Briefly, 1 nM ³H-mesulergine and varying concentrations of the compound are incubated with 5-HT_{2c} receptor membranes, following an incubation period, the membranes are washed and ³H-mesulergine bound to membranes is measured in a liquid scintillation counter. The amount of bound ³H-mesulergine at the varying concentration of competitor compound is used to derive the affinity (Kᵢ) of the compound for the 5-HT_{2c} receptor. 5-HT_{2c} receptor containing membranes are readily available from several companies including Perkin-Elmer and Euroscreen.

The following table shows Kᵢ (nM) binding data for selected compounds at both the MCH-1R and 5-HT_{2c} receptors.

TABLE V

| Compound | MCH-1R | 5-HT_{2C} |
|---|---|---|
| 6-(4-Chlorophenyl)-3-{(5)-6-[((5)-3-hydroxypyrrolidin-1- | 24 | 9552 |
| yl)methyl]-1,2,3,4-tetrahydronaphthalen-2-yl}thieno[3,2- | | |
| d]pyrimidin-4(3H)-one | | |
| 6-(4-Chlorophenyl)-3-{(S)-6-[(S)-2-hydroxypropylaminomethyl]- | 25 | 3080 |
| 1,2,3,4-tetrahydronapthealen-2-yl}thieno[3,2-d]pyrimidin- | | |
| 4(3H)-one | | |
| (S)-6-(4-Chlorophenyl)-3-[6-(morpholinomethyl)-1,2,3,4- | 103 | >10⁵ |
| tetrahydronapthalene-2-yl]thieno[3,2-d]pyrimidin-4-(3H)-one | | |
| (S)-6-(4-Chlorophenyl)-3-(6-{[4-(4-methylpiperazin-1-yl)piperidin- | 42 | 4792 |
| 1-yl]methyl}-1,2,3,4-tetrahydronapthalen-2-yl)thieno[3,2- | | |
| d]pyrimidin-4(3H)-one | | |
| (S)-6-(4-Chlorophenyl)-3-(6-((4-morpholinopiperidin-1-yl)methyl)- | 16 | >10⁵ |
| 1,2,3,4-tetrhaydronapthalene-2-yl)thieno[3,2,-d]pyrimidin- | | |
| 4-(3H)-one | | |
| (S)-6-(4-Chlorophenyl)-3-(6-diethylaminomethyl-1,2,3,4- | 13 | 6776 |
| tetrahydronaphthalen-2-yl)-4a,7a-dihydro-3H-thieno[3,2- | | |
| d]pyrimidin-4-one | | |
| (S)-6-(4-Chlorophenyl)-3-(6-((4-methylsulfonyl)piperazin-1- | 65 | >10⁵ |
| yl)methyl)-1,2,3,4-tetrahydronapthalen-2-yl)thieno[3,2,- | | |
| d]pyrimidin-4(3H)-one | | |
| (S)-3-(6-((4-Acetylpiperazin-1-yl)methyl)-1,2,3,4- | 35 | 14554 |
| tetrahydronapthalen-2-yl)-6-(4-chlorophenyl)thieno[3,2- | | |
| d]pyrimidine-4(3H)-one | | |
| (S)-6-(4-Chlorophenyl)-3-(6-piperidin-1-ylmethyl-1,2,3,4- | 24.3 | 3727 |
| tetrahydronaphthalen-2-yl)thieno[3,2-d]pyrimidin-4(3H)-one | | |
| (S)-6-(4-Chlorophenyl)-3-(6-pyrrolidin-1-ylmethyl-1,2,3,4- | 13.7 | 3727 |
| tetrahydronaphthalen-2-yl)thieno[3,2-d]pyrimidin-4(3H)-one | | |
| (S)-6-(4-Chlorophenyl)-3-[6-(1,1-dioxo-1λ⁶-thiomorpholin-4- | 1021 | >10⁵ |
| ylmethyl)-1,2,3,4-tetrahydronaphthalen-2-yl)thieno[3,2- | | |
| d]pyrimidin-4(3H)-one | | |
| (S)-6-(4-Chlorophenyl)-3-[6-(5-acetyl-2,5-diaza-bicyclo[2.2.1]hept- | 1.8 | >10⁵ |
| 2-ylmethyl)-1,2,3,4-tetrahydronaphthalen-2-yl)thieno[3,2- | | |
| d]pyrimidin-4(3H)-one | | |
| 6-(4-Chlorophenyl)-3-((S)-6-dimethylaminomethyl-1,2,3,4- | 7.4 | 3173 |
| tetrahydronapthealen-2-yl)thieno[3,2-d]pyrimidin-4(3H)-one | | |
| 6-(4-Chlorophenyl)-3-((S)-6-methylaminomethyl-1,2,3,4- | 0.4 | 2138 |
| tetrahydronapthealen-2-yl)thieno[3,2-d]pyrimidin-4(3H)-one | | |
| 6-(4-Chlorophenyl)-3-((S)-6-{[2- | 15.8 | >10⁵ |
| methanesulfonylethyl)methylamino]methyl}-1,2,3,4- | | |
| tetrahydronapthealen-2-yl)thieno[3,2-d]pyrimidin-4(3H)-one | | |
| 6-(4-Chlorophenyl)-3-{(S)-6-[(2-methanesulfonyl- | 22.6 | >10⁵ |
| ethylamino)methyl]-1,2,3,4-tetrahydronapthealen-2-yl}thieno[3,2- | | |
| d]pyrimidin-4(3H)-one | | |
| 6-(4-Chlorophenyl)-3-[(S)-6-(4-methanesulfonylpiperidin-1- | 8.2 | >10⁵ |
| ylmethyl)-1,2,3,4-tetrahydronapthealen-2-yl}thieno[3,2- | | |
| d]pyrimidin-4(3H)-one | | |
| 6-(4-Chlorophenyl)-3-{(S)-6-[(2-hydroxy-1- | 10.7 | >10⁵ |
| hydroxymethylethylamino)methyl]-1,2,3,4-tetrahydronapthealen-2- | | |
| yl}thieno[3,2-d]pyrimidin-4(3H)-one | | |
| (S)-3-[6-(4-Acetylpiperazin-l-ymethyl)-1,2,3,4- | 70.5 | >10⁵ |
| tetrahydronapthalen-2-yl]-6-(3-chlorophenyl)thieno[3,2- | | |
| d]pyrimidin-4(3H)-one | | |
| (S)-3-[6-(4-Acetylpiperazin-1-ymethyl)-1,2,3,4- | 17.9 | >10⁵ |
| tetrahydronapthalen-2-yl]-6-(3-fluorophenyl)thieno[3,2- | | |
| d]pyrimidin-4-(3H)one | | |
| (S)-3-[6-(4-Acetylpiperazin-1-ymethyl)-1,2,3,4- | 16.1 | 5462 |
| tetrahydronapthalen-2-yl]-6-(4-methylphenyl)thieno[3,2- | | |
| d]pyrimidin-4(3H)-one | | |
| (S)-3-[6-(4-Acetylpiperazin-1-ymethyl)-1,2,3,4- | 9.4 | 6358 |
| tetrahydronapthalen-2-yl]-6-(4-fluorophenyl)thieno[3,2- | | |
| d]pyrimidin-4(3H)-one | | |
| (S)-3-[6-(4-Acetylpiperazin-1-ymethyl)-1,2,3,4- | 988 | 8089 |
| tetrahydronapthalen-2-yl]-6-(pyridin-4-yl)thieno[3,2-d]pyrimidin- | | |
| 4(3H)-one | | |
| (S)-3-[6-(4-Acetylpiperazin-1-ymethyl)-1,2,3,4- | 54.5 | >10⁵ |
| tetrahydronapthalen-2-yl]-6-(3,4-dichlorophenyl)thieno[3,2- | | |
| d]pyrimidin-4(3H)-one | | |
| (S)-3-[6-(4-Acetylpiperazin-1-ymethyl)-1,2,3,4- | 36.6 | >10⁵ |
| tetrahydronapthalen-2-yl]-6-(3,4-difluorophenyl)thieno[3,2- | | |
| d]pyrimidin-4(3H)-one | | |
| (S)-3-[6-(4-Acetylpiperazin-1-ymethyl)-1,2,3,4- | 369.3 | >10⁵ |
| tetrahydronapthalen-2-yl]-6-(3,5-difluorophenyl)thieno[3,2- | | |
| d]pyrimidin-4(3H)-one | | |
| (S)-3-[6-(4-Acetylpiperazin-1-ymethyl)-1,2,3,4- | 8.4 | >10⁵ |
| tetrahydronapthalen-2-yl]-6-(2,4-difluorophenyl)thieno[3,2- | | |
| d]pyrimidin-4(3H)-one | | |
| (S)-3-[6-(4-Acetylpiperazin-1-ymethyl)-1,2,3,4- | 65.2 | >10⁵ |
| tetrahydronapthalen-2-yl]-6-(4-methoxyphenyl)thieno[3,2- | | |
| d]pyrimidin-4(3H)-one | | |
| (S)-3-[6-(4-Acetylpiperazin-1-ymethyl)-1,2,3,4- | 270 | 1764 |
| tetrahydronapthalen-2-yl]-6-(thien-2-yl)thieno[3,2-d]pyrimidin- | | |
| 4(3H)-one | | |
| (S)-3-[6-(4-Acetylpiperazin-1-ymethyl)-1,2,3,4- | 137 | 2013 |
| tetrahydronapthalen-2-yl]-6-(thien-3-yl)thieno[3,2-d]pyrimidin- | | |
| 4(3H)-one | | |
| (S)-3-[6-(4-Acetylpiperazin-1-ymethyl)-1,2,3,4- | 24.7 | 2372 |
| tetrahydronapthalen-2-yl]-6-(2,4dichlorophenyl)thieno[3,2- | | |
| d]pyrimidin-4(3H)-one | | |
| (S)-3-[6-(4-Acetylpiperazin-1-ymethyl)-1,2,3,4- | 34 | >10⁵ |
| tetrahydronapthalen-2-yl]-6-(4-cyanophenyl)thieno[3,2- | | |
| d]pyrimidin-4(3H)-one | | |
| (S)-4-(3-(6-((4-acetylpiperazin-1-yl)methyl)-1,2,3,4- | 397 | >10⁵ |
| tetrahydronaphthalen-2-yl)-4-oxo-3,4-dihydrothieno[3,2- | | |
| d]pyrimidin-6-yl)benzonitrile | | |
| (S)-3-[6-(4-Acetylpiperazin-1-ymethyl)-1,2,3,4- | 813 | >10⁵ |
| tetrahydronapthalen-2-yl]-6-(2-fluoropyridin-3-yl)thieno[3,2- | | |
| d]pyrimidin-4(3H)-one | | |
| (S)-3-[6-(4-Acetylpiperazin-1-ymethyl)-1,2,3,4- | 97 | >10⁵ |
| tetrahydronapthalen-2-yl]-6-(5-fluoropyridin-3-yl)thieno[3,2- | | |
| d]pyrimidin-4(3H)-one | | |
| (S)-3-[6-(4-Acetylpiperazin-1-ymethyl)-1,2,3,4- | 496 | >10⁵ |
| tetrahydronapthalen-2-yl]-6-(2-chloropyridin-4-yl)thieno[3,2- | | |
| d]pyrimidin-4(3H)-one | | |
| (S)-3-[6-(4-Acetylpiperazin-1-ymethyl)-1,2,3,4- | 1926 | >10⁵ |
| tetrahydronapthalen-2-yl]-6-(pyridin-3-yl)thieno[3,2-d]pyrimidin- | | |
| 4(3H)-one | | |
| (S)-3-[6-(4-Acetylpiperazin-1-ymethyl)-1,2,3,4- | 38 | >10⁵ |
| tetrahydronapthalen-2-yl]-6-(5-chloropyridin-3-yl)thieno[3,2- | | |
| d]pyrimidin-4(3H)-one | | |
| (S)-6-(Furan-3-yl)-3-[6-(methansulfonylpiperazin-1-ylmethyl)- | 733 | >10⁵ |
| 1,2,3,4-tetrahydro-naphthalen-2-yl]thieno[3,2-d]pyrimidin- | | |
| 4(3H)-one | | |
| (S)-6-(2,4-Dichlorophenyl)-3-[6-(methansulfonylpiperazin-1- | 107 | >10⁵ |
| ylmethyl)-1,2,3,4-tetrahydro-naphthalen-2-yl]thieno[3,2- | | |
| d]pyrimidin-4(3H)-one | | |
| (S)-6-(3-Cyanophenyl)-3-[6-(methansulfonylpiperazin-1-ylmethyl)- | 310 | >10⁵ |
| 1,2,3,4-tetrahydro-naphthalen-2-yl]thieno[3,2-d]pyrimidin- | | |
| 4(3H)-one | | |
| (S)-6-(Pyridin-3-yl)-3-[6-(methansulfonylpiperazin-1-ylmethyl)- | 752 | >10⁵ |
| 1,2,3,4-tetrahydro-naphthalen-2-yl]thieno[3,2-d]pyrimidin- | | |
| 4(3H)-one | | |
| (S)-6-(Thien-3-yl)-3-[6-(methansulfonylpiperazin-1-ylmethyl)- | 60 | >10⁵ |
| 1,2,3,4-tetrahydro-naphthalen-2-yl]thieno[3,2-d]pyrimidin- | | |
| 4(3H)-one | | |
| (S)-6-(5-Methoxypyridin-3-yl)-3-[6-(methansulfonylpiperazin-1- | 17.9 | >10⁵ |
| ylmethyl)-1,2,3,4-tetrahydro-naphthalen-2-yl]thieno[3,2- | | |
| d]pyrimidin-4(3H)-one | | |
| (S)-6-(2-Methoxypyridin-3-yl)-3-[6-(methansulfonylpiperazin-1- | 373 | >10⁵ |
| ylmethyl)-1,2,3,4-tetrahydro-naphthalen-2-yl]thieno[3,2- | | |
| d]pyrimidin-4(3H)-one | | |
| (S)-6-(5-Fluoropyridin-3-yl)-3-[6-(methansulfonylpiperazin-1- | 13.4 | >10⁵ |
| ylmethyl)-1,2,3,4-tetrahydro-naphthalen-2-yl]thieno[3,2- | | |
| d]pyrimidin-4(3H)-one | | |
| (S)-6-(5-Chloropyridin-3-yl)-3-[6-(methansulfonylpiperazin-1- | 21 | >10⁵ |
| ylmethyl)-1,2,3,4-tetrahydro-naphthalen-2-yl]thieno[3,2- | | |
| d]pyrimidin-4(3H)-one | | |
| (S)-6-(2-Chloropyridin-4-yl)-3-[6-(methansulfonylpiperazin-1- | 157 | >10⁵ |
| ylmethyl)-1,2,3,4-tetrahydro-naphthalen-2-yl]thieno[3,2- | | |
| d]pyrimidin-4(3H)-one | | |
| (S)-6-(3,4-Dichlorophenyl)-3-[6-(methansulfonylpiperazin-1- | 142 | >10⁵ |
| ylmethyl)-1,2,3,4-tetrahydro-naphthalen-2-yl]thieno[3,2- | | |
| d]pyrimidin-4(3H)-one | | |
| (S)-6-(Thien-2-yl)-3-[6-(methansulfonylpiperazin-1-ylmethyl)- | 122 | >10⁵ |
| 1,2,3,4-tetrahydro-naphthalen-2-yl]thieno[3,2-d]pyrimidin- | | |
| 4(3H)-one | | |
| (S)-6-(2,4-Difluorophenyl)-3-[6-(methansulfonylpiperazin-1- | 7.6 | >10⁵ |
| ylmethyl)-1,2,3,4-tetrahydro-naphthalen-2-yl]thieno[3,2- | | |
| d]pyrimidin-4(3H)-one | | |
| (S)-6-(3,4-Difluorophenyl)-3-[6-(methansulfonylpiperazin-1- | 39.3 | >10⁵ |
| ylmethyl)-1,2,3,4-tetrahydro-naphthalen-2-yl]thieno[3,2- | | |
| d]pyrimidin-4(3H)-one | | |
| (S)-6-(3,5-Difluorophenyl)-3-[6-(methansulfonylpiperazin-1- | 117 | >10⁵ |
| ylmethyl)-1,2,3,4-tetrahydro-naphthalen-2-yl]thieno[3,2- | | |
| d]pyrimidin-4(3H)-one | | |

All documents cited in the Detailed Description of the invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound having the formula: 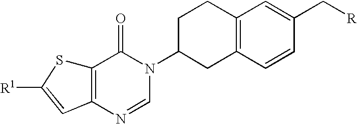
wherein:
R has the formula: 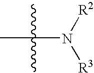
R² and R³ are independently chosen from:
i) hydrogen;
ii) C₁-C₄ substituted or unsubstituted alkyl; or
iii) R² and R³ are taken together to form a substituted or unsubstituted ring containing from 3 to 7 atoms;
R¹ is a unit chosen from:
i) C₆ or C₁₀ substituted or unsubstituted aryl ring; or
ii) C₃-C₅ substituted or unsubstituted heteroaryl rings.

2. A compound according to claim 1 wherein R² and R³ are taken together to form a ring containing from 3 to 7 atoms.

3. A compound according to claim 1 wherein R is chosen from substituted or unsubstituted pyrrolidin-1-yl, piperidin-1-yl, piperazin-1-yl, morpholin-4-yl, and 1,1-dioxo-1λ⁶-thiomorpholin-4-yl.

4. A compound according to claim 3, wherein R is substituted pyrrolidin-1-yl, 4-substituted piperidin-1-yl, 4-substituted piperazin-1-yl, 6-substituted 3,6-diazabicyclo[3.1.1]hept-3-yl, said substitution chosen from:
i) —NHCOR⁴;
ii) —COR⁴;
iii) C₁-C₄ linear, branched, or cyclic alkyl;
iv) —OR⁴;
v) —SO₂R⁴; and
vi) a heterocyclic ring chosen from pyrrolidin-1-yl, piperidin-1-yl, and morpholin-4-yl; and
R⁴ is hydrogen, methyl, ethyl, iso-propyl, and phenyl.

5. A compound according to claim 4, wherein R is chosen from 3-hydroxy-pyrrolidin-1-yl, 4-methylpiperazin-1-yl, 4-acetylpiperazin-1-yl, 4-methane-sulfonylpiperidin-1-yl, 4-methanesulfonylpiperazin-1-yl, 4-(morpholin-4-yl)piperazin-1-yl, and 6-methyl-3,6-diazabicyclo[3.1.1]hept-3-yl.

6. A compound according to claim 1 wherein R has the formula: 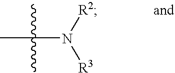
R² and R³ are independently chosen from hydrogen or substituted or unsubstituted methyl, ethyl, n-propyl, iso-propyl, n-butyl, sec-butyl, iso-butyl, and tert-butyl.

7. A compound according to claim 6 wherein R is chosen from
i) —NH₂;
ii) —NHCH₃;
iii) —N(CH₃)₂;
iv) —NHCH₂CH₃;
v) —N(CH₃)(CH₂CH₃); and
vi) —N(CH₂CH₃)₂.

8. A compound according to claim 6 wherein R³ is hydrogen or methyl and R² is chosen from:
i) —CH₂CH₂OH;
ii) —CH₂CH₂CH₂OH;
iii) —CH₂CH₂CH₂CH₂OH;
iv) —CH₂CH(OH)CH₃;
v) —CH₂CH(OH)CH₂CH₃;
vi) —CH₂CH₂CH(OH)CH₃; and
vii) —CH(CH₂OH)₂.

9. A compound according to claim 1, wherein R¹ is phenyl or substituted phenyl, said substitutions chosen from one or more:
i) C₁-C₄ linear or branched alkyl;
ii) C₁-C₄ linear or branched alkoxy; and
iii) halogen.

10. A compound according to claim 9, wherein R¹ is chosen from 2-fluorophenyl, 3-fluorophenyl, 4-fluorophenyl, 2,3-difluorophenyl, 2,4-difluorophenyl, 2,5-difluorophenyl, 2,6-difluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 2-cyanophenyl, 3-cyanophenyl, 4-cyanophenyl, 2,3-dicyanophenyl, 2,4-dicyanophenyl, 2,5-dicyanophenyl, 2,6-dicyanophenyl, 2-methylphenyl, 3-methylphenyl, 4-methylphenyl, 2,3-dimethylphenyl, 2,4-dimethylphenyl, 2,5-dimethylphenyl, and 2,6-dimethylphenyl.

11. A compound according to claim 1, wherein R¹ is a substituted or unsubstituted C₄ or C₅ heteroaryl, said substitutions chosen from one or more:
i) C₁-C₄ linear or branched alkyl;
ii) C₁-C₄ linear or branched alkoxy; and
iii) halogen.

12. A compound according to claim 11, wherein R¹ is chosen from furan-2-yl, furan-3-yl, thiophene-2-yl, and thiophene-3-yl.

13. A compound according to claim 11, wherein R¹ is chosen from pyridin-3-yl, 2-fluoropyridin-3-yl, 6-fluoropyridin-3-yl, 2-chloropyridin-3-yl, 6-chloropyridin-3-yl, 2-methoxypyridin-3-yl, 6-methoxypyridin-3-yl, 2-methylpyridin-3-yl, 6-methylpyridin-3-yl, pyridin-4-yl, 2-fluoropyridin-4-yl, 6-fluoropyridin-4-yl, 2-chloropyridin-4-yl, 6-chloropyridin-4-yl, 2-methoxypyridin-4-yl, 6-methoxypyridin-4-yl, 2-methylpyridin-4-yl, and 6-methylpyridin-4-yl.

14. A compound according to claim 1, wherein R¹ is 4-fluorophenyl or 4-chloro-phenyl.

15. A compound having the formula: 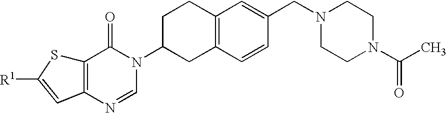
wherein R¹ is chosen from:
i) C₆ or C₁₀ substituted or unsubstituted aryl ring; or
ii) C₃-C₅ substituted or unsubstituted heteroaryl rings.

16. A compound according to claim 15, wherein R¹ is phenyl or substituted phenyl, said substitutions chosen from one or more:
i) C₁-C₄ linear or branched alkyl;
ii) C₁-C₄ linear or branched alkoxy; and
iii) halogen.

17. A compound according to claim 16, wherein R¹ is chosen from 2-fluorophenyl, 3-fluorophenyl, 4-fluorophenyl, 2,3-difluorophenyl, 2,4-difluorophenyl, 2,5-difluorophenyl, 2,6-difluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 2-cyanophenyl, 3-cyanophenyl, 4-cyanophenyl, 2,3-dicyanophenyl, 2,4-dicyanophenyl, 2,5-dicyanophenyl, 2,6-dicyanophenyl, 2-methylphenyl, 3-methylphenyl, 4-methylphenyl, 2,3-dimethylphenyl, 2,4-dimethylphenyl, 2,5-dimethylphenyl, and 2,6-dimethylphenyl.

18. A compound according to claim 15, wherein R¹ is a substituted or unsubstituted C₄ or C₅ heteroaryl, said substitutions chosen from one or more:
i) C₁-C₄ linear or branched alkyl;
ii) C₁-C₄ linear or branched alkoxy; and
iii) halogen.

19. A compound according to claim 18, wherein R¹ is chosen from furan-2-yl, furan-3-yl, thiophene-2-yl, and thiophene-3-yl.

20. A compound according to claim 18, wherein R¹ is chosen from pyridin-3-yl, 2-fluoropyridin-3-yl, 6-fluoropyridin-3-yl, 2-chloropyridin-3-yl, 6-chloropyridin-3-yl, 2-methoxypyridin-3-yl, 6-methoxypyridin-3-yl, 2-methylpyridin-3-yl, 6-methylpyridin-3-yl, pyridin-4-yl, 2-fluoropyridin-4-yl, 6-fluoropyridin-4-yl, 2-chloropyridin-4-yl, 6-chloropyridin-4-yl, 2-methoxypyridin-4-yl, 6-methoxypyridin-4-yl, 2-methylpyridin-4-yl, and 6-methylpyridin-4-yl.

21. A compound according to claim 15, wherein R¹ is 4-fluorophenyl or 4-chloro-phenyl.

22. A compound having the formula: 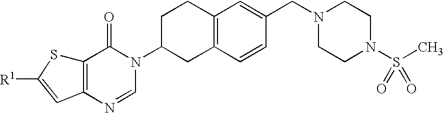
wherein R¹ is chosen from:
i) C₆ or C₁₀ substituted or unsubstituted aryl ring; or
ii) C₃-C₅ substituted or unsubstituted heteroaryl rings.

23. A compound according to claim 22 wherein R¹ is phenyl or substituted phenyl, said substitutions chosen from one or more:
i) C₁-C₄ linear or branched alkyl;
ii) C₁-C₄ linear or branched alkoxy; and
iii) halogen.

24. A compound according to claim 23, wherein R¹ is chosen from 2-fluorophenyl, 3-fluorophenyl, 4-fluorophenyl, 2,3-difluorophenyl, 2,4-difluorophenyl, 2,5-difluorophenyl, 2,6-difluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 2-cyanophenyl, 3-cyanophenyl, 4-cyanophenyl, 2,3-dicyanophenyl, 2,4-dicyanophenyl, 2,5-dicyanophenyl, 2,6-dicyanophenyl, 2-methylphenyl, 3-methylphenyl, 4-methylphenyl, 2,3-dimethylphenyl, 2,4-dimethylphenyl, 2,5-dimethylphenyl, and 2,6-dimethylphenyl.

25. A compound according to claim 22, wherein R¹ is a substituted or unsubstituted C₄ or C₅ heteroaryl, said substitutions chosen from one or more:
i) C₁-C₄ linear or branched alkyl;
ii) C₁-C₄ linear or branched alkoxy; and
iii) halogen.

26. A compound according to claim 25, wherein R¹ is chosen from furan-2-yl, furan-3-yl, thiophene-2-yl, and thiophene-3-yl.

27. A compound according to claim 25, wherein R¹ is chosen from pyridin-3-yl, 2-fluoropyridin-3-yl, 6-fluoropyridin-3-yl, 2-chloropyridin-3-yl, 6-chloropyridin-3-yl, 2-methoxypyridin-3-yl, 6-methoxypyridin-3-yl, 2-methylpyridin-3-yl, 6-methylpyridin-3-yl, pyridin-4-yl, 2-fluoropyridin-4-yl, 6-fluoropyridin-4-yl, 2-chloropyridin-4-yl, 6-chloropyridin-4-yl, 2-methoxypyridin-4-yl, 6-methoxypyridin-4-yl, 2-methylpyridin-4-yl, and 6-methylpyridin-4-yl.

28. A compound according to claim 22, wherein R¹ is 4-fluorophenyl or 4-chloro-phenyl.

29. A compound chosen from:
(S)-6-(4-Chlorophenyl)-3-(6-diethylaminomethyl-1,2,3,4-tetrahydronaphthalen-2-yl)thieno[3,2-d]pyrimidin-4(3H)-one;
(S)-6-(4-Chlorophenyl)-3-(6-((4-morpholinopiperidin-1-yl)methyl)-1,2,3,4-tetrhaydronapthalene-2-yl)thieno[3,2,-d]pyrimidin-4-(3H)-one;
(S)-6-(4-Chlorophenyl)-3-(6-{[4-(4-methylpiperazin-1-yl)piperidin-1-yl]methyl}-1,2,3,4-tetrahydronapthalen-2-yl)thieno[3,2-d]pyrimidin-4(3H)-one;
(S)-6-(4-Chlorophenyl)-3-(6-((4-methylsulfonyl)piperazin-1-yl)methyl)-1,2,3,4-tetrahydronapthalen-2-yl)thieno[3,2,-d]pyrimidin-4(3H)-one;
(S)-6-(Chlorophenyl)-3-[6-(morpholinomethyl)-1,2,3,4-tetrahydronapthalene-2-yl]thieno[3,2-d]pyrimidin-4-(3H)-one;
(S)-3-(6-((4-Acetylpiperazin-1-yl)methyl)-1,2,3,4-tetrahydronapthalen-2-yl)-6-(4-chlorophenyl)thieno[3,2-d]pyrimidine-4(3H)-one;
6-(4-Chlorophenyl)-3-{(S)-6-[((S)-3-hydroxypyrrolidin-1-yl)methyl]-1,2,3,4-tetrahydronaphthalen-2-yl}thieno[3,2-d]pyrimidin-4(3H)-one;
6-(4-Chlorophenyl)-3-((S)-6-[((S)-2-hydroxypropylaminomethyl)-1,2,3,4-tetrahydronapthealen-2-yl]thieno[3,2-d]pyrimidin-4(3H)-one;
(S)-6-(4-Chlorophenyl)-3-(6-(piperidin-1-ylmethyl)-1,2,3,4-tetrahydronaphthalen-2-yl)thieno[3,2-d]pyrimidin-4(3H)-one;
(S)-6-(4-Chlorophenyl)-3-(6-(pyrrolidin-1-ylmethyl)-1,2,3,4-tetrahydro-naphthalen-2-yl)thieno[3,2-d]pyrimidin-4(3H)-one;
(S)-3-{6-[(4-Acetylpiperazin-1-yl)methyl]-1,2,3,4-tetrahydro-naphthalen-2-yl}-fluorophenyl)thieno[3,2-d]pyrimidin-4(3H)-one;
3-{(S)-6-[(4-Acetylpiperazin-1-yl)methyl]-1,2,3,4-tetrahydronaphthalen-2-yl}-6-(2-fluorophenyl)thieno[3,2-d]pyrimidin-4(3H)-one;
3-{(S)-6-[(4-Acetylpiperazin-1-yl)methyl]-1,2,3,4-tetrahydronaphthalen-2-yl}-6-(3-fluorophenyl)thieno[3,2-d]pyrimidin-4(3H)-one;
(S)-3-{6-[(4-Acetylpiperazin-1-yl)methyl]-1,2,3,4-tetrahydronaphthalen-2-yl}-6-(3,4-difluorophenyl)thieno[3,2-d]pyrimidin-4(3H)-one;
(S)-3-{6-[(4-Acetylpiperazin-1-yl)methyl]-1,2,3,4-tetrahydronaphthalen-2-yl}-6-(3,5-difluorophenyl)thieno[3,2-d]pyrimidin-4(3H)-one;
(S)-6-(3,4-Difluorophenyl)-3-{6-[(4-(methylsulfonyl)piperazin-1-yl]methyl}-1,2,3,4-tetrahydronaphthalen-2-yl)thieno[3,2-d]pyrimidin-4(3H)-one;
(S)-3-{6-[(4-Acetylpiperazin-1-yl)methyl]-1,2,3,4-tetrahydronaphthalen-2-yl}-6-(2,4-difluorophenyl)thieno[3,2-d]pyrimidin-4(3H)-one;
(S)-6-(2,4-Difluorophenyl)-3-{6-[(4-(methylsulfonyl)piperazin-1-yl]methyl}-1,2,3,4-tetrahydro-naphthalen-2-yl)thieno[3,2-d]pyrimidin-4(3H)-one;
(S)-6-(3,5-Difluorophenyl)-3-(6-{[4-(methylsulfonyl)piperazin-1-yl]methyl}-1,2,3,4-tetrahydronaphthalen-2-yl)thieno[3,2-d]pyrimidin-4(3H)-one;
3-{(S)-6-[(4-Acetylpiperazin-1-yl)methyl]-1,2,3,4-tetrahydronaphthalen-2-yl}-6-(3-chlorophenyl)thieno[3,2-d]pyrimidin-4(3H)-one;
(S)-3-{6-[(4-Acetylpiperazin-1-yl)methyl]-1,2,3,4-tetrahydronaphthalen-2-yl}-6-(2,4-dichlorophenyl)thieno[3,2-d]pyrimidin-4(3H)-one;
(S)-6-(2,4-Dichlorophenyl)-3-{6-[(4-(methylsulfonyl)piperazin-1-yl]methyl}-1,2,3,4-tetrahydronaphthalen-2-yl)thieno[3,2-d]pyrimidin-4(3H)-one;
(S)-3-{6-[(4-Acetylpiperazin-1-yl)methyl]-1,2,3,4-tetrahydronaphthalen-2-yl}-6-(3,4-dichlorophenyl)thieno[3,2-d]pyrimidin-4(3H)-one;
(S)-6-(3,4-Dichlorophenyl)-3-{6-[(4-(methylsulfonyl)piperazin-1-yl]methyl}-1,2,3,4-tetrahydronaphthalen-2-yl)thieno[3,2-d]pyrimidin-4(3H)-one;
(S)-4-(3-{6-[(4-Acetylpiperazin-1-yl)methyl]-1,2,3,4-tetrahydronaphthalen-2-yl}-4-oxo-3,4-dihydrothieno[3,2-d]pyrimidin-6-yl)benzonitrile;
(S)-3-(3-{6-[(4-(Methylsulfonyl)piperazin-1-yl]methyl}-1,2,3,4-tetrahydro-naphthalen-2-yl)-4-oxo-3,4-dihydrothieno[3,2-d]pyrimidin-6-yl)benzonitrile;
(S)-3-(3-{6-[(4-Acetylpiperazin-1-yl)methyl]-1,2,3,4-tetrahydronaphthalen-2-yl}-4-oxo-3,4-dihydrothieno[3,2-d]pyrimidin-6-yl)benzonitrile;
(S)-3-{6-[(4-Acetylpiperazin-1-yl)methyl]-1,2,3,4-tetrahydronaphthalen-2-yl}-6-p-tolylthieno[3,2-d]pyrimidin-4(3H)-one;
(S)-3-{6-[(4-Acetylpiperazin-1-yl)methyl)-1,2,3,4-tetrahydronaphthalen-2-yl)-6-(pyridin-3-yl)thieno[3,2-d]pyrimidin-4(3H)-one;
(S)-3-{6-[(4-(Methylsulfonyl)piperazin-1-yl]methyl}-1,2,3,4-tetrahydro-naphthalen-2-yl)-6-(pyridin-3-yl)thieno[3,2-d]pyrimidin-4(3H)-one;
(S)-3-{6-[(4-Acetylpiperazin-1-yl)methyl]-1,2,3,4-tetrahydronaphthalen-2-yl}-6-(pyridin-4-yl)thieno[3,2-d]pyrimidin-4(3H)-one;
(S)-3-{6-[(4-(Methylsulfonyl)piperazin-1-yl]methyl}-1,2,3,4-tetrahydro-naphthalen-2-yl)-6-(pyridin-4-yl)thieno[3,2-d]pyrimidin-4(3H)-one;
(S)-3-(6-((4-Acetylpiperazin-1-yl)methyl)-1,2,3,4-tetrahydronaphthalen-2-yl)-6-(2-fluoropyridin-3-yl)thieno[3,2-d]pyrimidin-4(3H)-one;
(S)-3-{6-[(4-Acetylpiperazin-1-yl)methyl]-1,2,3,4-tetrahydronaphthalen-2-yl}-6-(6-fluoropyridin-3-yl)thieno[3,2-d]pyrimidin-4(3H)-one;
(S)-6-(6-Fluoropyridin-3-yl)-3-{6-[(4-(methylsulfonyl)piperazin-1-yl]methyl}-1,2,3,4-tetrahydronaphthalen-2-yl)thieno[3,2-d]pyrimidin-4(3H)-one;
(S)-3-{6-[(4-Acetylpiperazin-1-yl)methyl]-1,2,3,4-tetrahydronaphthalen-2-yl}-6-(6-chloropyridin-3-yl)thieno[3,2-d]pyrimidin-4(3H)-one;
(S)-3-{6-[(4-Acetylpiperazin-1-yl)methyl]-1,2,3,4-tetrahydronaphthalen-2-yl}-6-(2-chloropyridin-4-yl)thieno[3,2-d]pyrimidin-4(3H)-one;
(S)-6-(6-Chloropyridin-3-yl)-3-{6-[(4-(methylsulfonyl)piperazin-1-yl]methyl}-1,2,3,4-tetrahydronaphthalen-2-yl)thieno[3,2-d]pyrimidin-4(3H)-one;
(S)-6-(2-Chloropyridin-4-yl)-3-{6-[(4-(methylsulfonyl)piperazin-1-yl]methyl}-1,2,3,4-tetrahydronaphthalen-2-yl)thieno[3,2-d]pyrimidin-4(3H)-one;
(S)-3-{6-[(4-Acetylpiperazin-1-yl)methyl]-1,2,3,4-tetrahydronaphthalen-2-yl}-6-(2-methoxypyridin-3-yl)thieno[3,2-d]pyrimidin-4(3H)-one;
(S)-6-(6-Methoxypyridin-3-yl)-3-{6-[(4-(methylsulfonyl)piperazin-1-yl]methyl}-1,2,3,4-tetrahydronaphthalen-2-yl)thieno[3,2-d]pyrimidin-4(3H)-one;
(S)-3-{6-[(4-Acetylpiperazin-1-yl)methyl]-1,2,3,4-tetrahydronaphthalen-2-yl}-6-(6-methoxypyridin-3-yl)thieno[3,2-d]pyrimidin-4(3H)-one;
(S)-6-(2-Methoxypyridin-3-yl)-3-{6-[(4-(methylsulfonyl)piperazin-1-yl]methyl}-1,2,3,4-tetrahydronaphthalen-2-yl)thieno[3,2-d]pyrimidin-4(3H)-one;
(S)-3-{6-[(4-Acetylpiperazin-1-yl)methyl]-1,2,3,4-tetrahydronaphthalen-2-yl}-6-(thiophen-2-yl)thieno[3,2-d]pyrimidin-4(3H)-one:
(S)-3-{6-[(4-Acetylpiperazin-1-yl)methyl]-1,2,3,4-tetrahydronaphthalen-2-yl}-6-(thiophen-3-yl)thieno[3,2-d]pyrimidin-4(3H)-one;
(S)-3-{6-[(4-(Methylsulfonyl)piperazin-1-yl]methyl}-1,2,3,4-tetrahydro-naphthalen-2-yl)-6-(thiophen-3-yl)thieno[3,2-d]pyrimidin-4(3H)-one;
(S)-3-{6-([4-(Methylsulfonyl)piperazin-1-yl]methyl}-1,2,3,4-tetrahydro-naphthalen-2-yl)-6-(thiophen-2-yl)thieno[3,2-d]pyrimidin-4(3H)-one;
(S)-3-{6-[(4-Acetylpiperazin-1-yl)methyl]-1,2,3,4-tetrahydronaphthalen-2-yl}-6-(furan-3-yl)thieno[3,2-d]pyrimidin-4(3H)-one;
(S)-6-(Furan-3-yl)-3-{6-[(4-(methylsulfonyl)piperazin-1-yl]methyl}-1,2,3,4-tetrahydronaphthalen-2-yl)thieno[3,2-d]pyrimidin-4(3H)-one.

30. A composition comprising:
a) a compound according to claim 1; and
b) one or more pharmaceutically acceptable excipients.

31. A method for controlling obesity in humans and higher mammals comprising administering to a human a composition comprising a compound according to claim 1.

32. A composition comprising:
a) a compound according to claim 29; and
b) one or more pharmaceutically acceptable excipients.

33. A method for controlling obesity in humans and higher mammals comprising administering to a human a composition comprising a compound according to claim 29.
